(19) United States
(12) Patent Application Publication
(10) Pub. No.: US 2007/0043034 A1
(43) Pub. Date: Feb. 22, 2007

(54) Chlorine Ion Uptake Modulators and Uses Thereof

(76) Inventors: Kevin Staley, Denver, CO (US); Volodya Dzhala, Denver, CO (US); Frances E. Jensen, Chestnut Hill, MA (US); Roderic Smith, Anchorage, AK (US)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(21) Appl. No.: 11/464,977

(22) Filed: Aug. 16, 2006

Related U.S. Application Data
(60) Provisional application No. 60/708,983, filed on Aug. 16, 2005.

(51) Int. Cl. A61K 31/5513; A61K 31/549; A61K 31/515; A61K 31/195

(52) U.S. Cl. 514/221; 514/223.2; 514/471; 514/563; 514/562; 514/270

(57) ABSTRACT

The present invention provides methods for using chloride ion uptake modulators to treat disorders that are mediated by a sodium/potassium/chloride cotransporter.

### CROSS-REFERENCE TO RELATED APPLICATIONS

This application claims the priority benefit of U.S. Provisional Application No. 60/708,983, filed Aug. 16, 2005, which is incorporated herein by reference in its entirety.

### FEDERALLY FUNDED RESEARCH

The U.S. Government has a paid-up license in this invention and the right in limited circumstances to require the patent owner to license others on reasonable terms as provided for by the terms of Grant No. NS31718 and NIH/NINDS RO1 NS 40109-04 awarded by the National Institutes of Health.

### FIELD OF THE INVENTION

The present invention relates to chloride ion uptake modulators and methods for using the same.

### BACKGROUND OF THE INVENTION

Epilepsy and seizures affect 2.5 million Americans of all ages. Direct and indirect costs of treatment for epilepsy and seizures are estimated to be $12.5 billion annually. It is believed that ten percent of the American population will experience a seizure in their lifetime. Although seizures are generally associated with epilepsy, seizures can, and often do occur in the absence of epilepsy. For example, neonatal seizures represent an age-specific seizure disorder that is usually considered to be in a separate category from epilepsy. It is believed that approximately 1% of all neonates experience seizures during the neonatal period, generally defined as the first month of life. Although seizures often occur in the absence of another neurological disorder, neonatal seizures frequently are a sign of an underlying disease. Neonatal seizures also have many other characteristics that are quite different from seizures in children and adults.

Seizures are relatively common in the neonatal period. Although the clinical manifestations of neonatal seizures may be suppressed by current treatments, in some instances electroencephalograms (EEG) recordings have demonstrated ongoing cortical seizure activity in neonates. The efficacy of current treatments using antiepileptic drugs in neonatal seizures using two different antiepileptic drugs revealed equal efficacy, each stopping neonatal seizures in only about 45% of patients. The combined therapy resulted in about 60% of the seizures controlled, leaving 40% of the patients where seizures were not adequately controlled using this therapy. Uncontrolled, these seizures can lead to permanent debilitating problems, brain damage, or even death.

General principles of treating seizures in neonates have been similar to those principles of treating seizures in children and adults. But, there are some important differences in neonatal seizures compared to seizures in children and adults. Like seizures in more mature patients, if there is a treatable cause for the neonatal seizures, such as hypoglycemia, infection, or intracranial hemorrhage, then the underlying etiology should be treated with the appropriate therapy. However, in some cases, there is no known underlying cause that can be treated, or the underlying cause is not known with sufficient certainty to warrant treatment of that cause.

In cases where an underlying cause cannot be treated, neonatal seizures are often treated directly with drugs typically used to treat children and adult seizures. Traditionally, the repertoire of drugs used for neonatal seizures is relatively limited due in part to the fact that few drugs have been formally tested in the neonatal population. In addition, the complicated metabolism and pharmacokinetics of neonates makes use of some drugs difficult.

Accordingly, there is a continuing need for new therapies for seizures generally, and neonatal seizures in particular.

### SUMMARY OF THE INVENTION

The invention provides methods for using diuretic compounds to treat a various disorders, in particular sodium potassium chloride cotransport mediated disorders, and disorders associated with excitotoxicity in the brain that is exacerbated by impaired inhibition of γ-aminobutyric acid.

In one aspect, the invention provides a method for treating a sodium potassium chloride cotransport mediated disorder in a subject. The method generally comprises administering to a subject in need of such a treatment, a therapeutically effective amount of a diuretic compound.

In some embodiments, the sodium potassium chloride cotransport mediated disorder is seizure, epilepsy, trauma, or a disease associated with a hypoxic-ischemic event. Within these embodiments, in some instances the sodium potassium chloride cotransport mediated disorder is a neonatal seizure, acute seizure, a chronic epilepsy, stroke, trauma, cortical malformation, CNS tumor or metabolic disorder.

In many cases, the chronic epilepsy that is treated by methods of the invention is a chronic temporal lobe epilepsy, or chronic epilepsy related to stroke, metabolic disorder, trauma, malformation of cortical development or tumor.

While a variety of diuretic compounds are suitable for methods of the invention, in some embodiments the diuretic compound is 3-(aminosulfonyl)-5-(butylamino)-4-phenoxybenzoic acid, 5-(aminosulfonyl)-4-chloro-2-[(2-furanylmethyl)-amino]benzoic acid, [2,3-dichloro-4-(2-methylene-1-oxobutyl) phenoxy]acetic acid, or a combination thereof. Other diuretic compounds are well known to one skilled in the art and are generally disclosed in Physician's Desk Reference 60^{th} Ed., Medical Economics Co. Inc., 2006, Montvale, N.J. In one particular embodiment, the diuretic compound is 3-(aminosulfonyl)-5-(butylamino)-4-phenoxybenzoic acid.

In other embodiments, the method further comprises administering a second therapeutic agent, wherein the second therapeutic agent comprises a γ-aminobutyric acid A (GABA_{A}) receptor modulator, an anticonvulsant agent, ion channel inactivator, an antidiuretic agent, or a combination thereof. In some instances, the GABA_{A} receptor modulator comprises a GABA_{A} receptor positive allosteric modulator. Suitable GABA_{A} receptor positive allosteric modulators include barbiturates, benzodiazepines, and a combination thereof.

In other embodiments, the GABA_{A} receptor modulator is an anticonvulsant agent. In some instances, the anticonvulsant GABA_{A} receptor modulator is tiagabine, acetazolamide, or a combination thereof.

Still in other embodiments, the antidiuretic agent is a peripherally-acting antidiuretic agent.

In some embodiments, methods of the invention are used to treat human subjects. In particular, neonates.

Another aspect of the invention provides a method for treating a sodium potassium chloride cotransport mediated disorder comprising administering to a subject in need of such a treatment a therapeutically effective amount of a compound capable of decreasing neuronal chloride accumulation in the subject.

In some embodiments, the compound capable of decreasing neuronal chloride accumulation comprises 3-(aminosulfonyl)-5-(butylamino)-4-phenoxybenzoic acid, 5-(aminosulfonyl)-4-chloro-2-[(2-furanylmethyl)amino] benzoic acid, [2,3-dichloro-4-(2-methylene-1-oxobutyl) phenoxy]acetic acid, or a combination thereof.

Still in other embodiments, methods of the invention comprises administering a second therapeutic agent. Typically, the second therapeutic agent comprises a γ-aminobutyric acid A (GABA_{A}) receptor modulator, an anticonvulsant agent, ion channel modulator, an antidiuretic agent, or a combination thereof.

In some instances, the GABA_{A} receptor modulator comprises a GABA_{A} receptor positive allosteric modulator.

Yet another aspect of the invention provides a method for treating a neonatal seizure. Generally, such method comprises administering to a subject in need of such a treatment a therapeutically effective amount of:
(i) a compound capable of decreasing neuronal chloride accumulation;
(ii) a compound capable of modulating GABA production or modulating GABA_{A} receptor activity; or
(iii) a combination thereof.

In some embodiments, the compound capable of decreasing neuronal chloride accumulation comprises 3-(aminosulfonyl)-5-(butylamino)-4-phenoxybenzoic acid, 5-(aminosulfonyl)-4-chloro-2-[(2-furanylmethyl)amino] benzoic acid, [2,3-dichloro-4-(2-methylene-1-oxobutyl) phenoxy]acetic acid, or a combination thereof.

Yet in other embodiments, method further comprises administering a second therapeutic reagent. A suitable second therapeutic reagent for methods of the invention comprises an anticonvulsant agent, an antidiuretic agent, or a combination thereof. Within these embodiments, in some instances the second therapeutic reagent composition comprises an anticonvulsant agent.

Still another aspect of the invention provides a method for treating a disorder mediated by excitotoxicity in the brain that is exacerbated by impaired inhibition of γ-aminobutyric acid (GABA). The method generally comprises administering to a subject in need of such a treatment, a therapeutically effective amount of a diuretic compound.

Often such method is used to treat the disorder such as epilepsy, seizure, or a disease associated with a hypoxic-ischemic event.

In some embodiments, the method often comprises administering a second therapeutic agent. Typically, the second therapeutic agent comprises a GABA receptor modulator, an anticonvulsant agent, an antidiuretic agent, or a combination thereof. In some instances, the GABA receptor modulator is a GABA agonist.

Another aspect of the present invention provides a method for treating a sodium potassium chloride cotransport mediated disorder in children and adult subjects as well as other mammalian subjects.

### BRIEF DESCRIPTION OF THE DRAWINGS

FIGS. 1A-1C represent some of the effects of inhibition of the sodium/potassium/chloride NKCCl cotransporter on GABA (γ-aminobutyric acid) excitability.

FIGS. 2A-2D represent the effects of phenobarbital on epileptiform activity in an in vitro assay system using neonatal rat hippocampus.

FIGS. 3A-3D represent the effects of bumetanide on epileptiform activity in an in vitro assay system using neonatal rat hippocampus.

FIGS. 4A-4D represents in vivo activity of seizures induced in rats exposed to kainic acid (KA).

FIGS. 5A-5C represent in a) control in vivo activity of seizures induced in rats exposed to KA compared to in vivo activity of seizures induced in rats exposed to KA then exposed to b) phenobarbital or c) bumetanide.

FIGS. 6A-6D represents NKCCl vs. KCC2 expression in human and rat cortex.

FIGS. 7A-7E are various graphs showing that NKCCl activity was required to maintain elevated Clᵢ.

FIG. 8A is a graph showing that after an outward Cl^{e} transient, Clᵢ returned to steady state via NKCCl transport and dendritic diffusion.

FIG. 8B is a graph showing Clᵢ as a function of time after an outward Cl⁻ transient for control (filled circles) and with NKCCl blocked (open circles) for a single neuron; each was fit to a single exponential.

FIG. 8C is a graph of the m of NKCCl Cl⁻ transport which was determined by subtracting first order rate constants (k=1/r).

FIG. 8D is a graph showing that following dendritic Cl⁻ efflux, NKCCl returned Clᵢ to steady state by inward Cl⁻ transport (n=5). Following dendritic Cl⁻ influx, NKCCl returned Clᵢ to steady state via outward Cl⁻ transport (n=3).

FIG. 9A is a graph showing that predicted Clᵢ for NKCCl at thermodynamic equilibrium correlated with previously reported transport stoichiometries and Clᵢ.

FIG. 9B is a graph showing NKCCl mediated inward Cl⁻ transport (same data as in FIG. 8D: filled circles, mean±s.e.m.), with the transport velocity (ν) calculated according to Michaelis-Menten kinetics (MM, dashed line), or as the product of a Michaelis-Menten conductance term (ν_{MM}) and a driving force term (ΔG/ΔGₜ₌₀) for various Na⁺:K⁺:Cl⁻ transport stoichiometries (ratios 2:1:3, 1:1:2, 1:2:3, and 1:3:4, dotted lines; 1:4:5, solid line).

FIG. 9C is a graph showing normalized driving force (left ordinate) and transport velocity (right ordinate) as a function of time for each Cl⁻ transport stoichiometry.

FIG. 9D is a graphs showing NKCCl mediated Cl⁻ transport with pipette [Na⁺] (Naₚᵢₚₑₜₜₑ)=0 mM (filled circles, n=5) or 9 mM (filled squares, n=3).

FIG. 9E is a graph showing that the slow initial velocity and τ of NKCCl Cl⁻ transport with 9 mM Naₚᵢₚₑₜₜₑ can be accounted for by a transient decrease in the free energy available for transport (FIG. 9B) that arises from a Naᵢ transient that resolves with a time constant of 1.85 s.

FIG. 9F is a graph showing that with 9 mM Naₚᵢₚₑₜₜₑ (squares, R²=1×10⁻⁶) NKCCl Cl⁻ transport rate was independent of ΔClᵢ and with 0 mM Naₚᵢₚₑₜₜₑ (circles, R²-0.85), larger Clᵢ depletions correlated with slower NKCCl Cl⁻ transport.

FIG. 10A is a graph showing that following a train of action potentials in the recorded cell (20 Hz, 2.5 min), Clᵢ reached a new steady state.

FIG. 10B is a graph showing Cl⁻ transport rate as a function of the magnitude of Cl⁻ depletion before and after postsynaptic action potentials for the cell in FIG. 10A.

FIG. 10C is a graph showing that repetitive postsynaptic spiking had no effect on Cl⁻ transport when the experiment was repeated in the presence of 10 μM dihydroouabain (DHO), a selective Na⁺—K⁺-ATPase inhibitor.

FIG. 10D is a graph in which Clᵢ was normalized to value before action potentials.

FIG. 11A is a graph showing that continuous superfusion of low-Mg²⁺ ACSF resulted in recurrent tonic-clonic epileptiform activity.

FIG. 11B is a graph showing that inter-seizure intervals (ISI) gradually decreased following continuous application of low-Mg²⁺-ACSF.

FIG. 11C is a graph showing that power of recurrent seizures gradually increased following continuous application of low-Mg²⁺-ACSF.

FIG. 12A is a graph showing extracellular field potential recording in the CA3 pyramidal cell layer in the intact hippocampus of a P5 rat. Continuous application of low-Mg²⁺ ACSF induced recurrent tonic-clonic seizures.

FIG. 12B shows a graph of inter-seizure intervals before, during and after phenobarbital application.

FIG. 12C is a graph showing power of recurrent seizures before, during and after phenobarbital application.

FIG. 13A is a graph of extracellular field potential recording in the CA3 pyramidal cell layer in the intact hippocampus of a P5 rat.

FIG. 13B is a graph showing inter-seizure intervals before, during and after application of drugs.

FIG. 13C is a graph showing power of recurrent seizures before, during and after simultaneous application of phenobarbital and bumetanide.

FIG. 14A shows a graph for mean frequency of recurrent seizures in control low-Mg²⁺ ACSF recordings (n=10) and before, during and after drug applications (n=10 for each drug or combination of drugs tested).

FIG. 14B shows a graph of mean power of extracellular field potential activity over 2-hour windows in control low-Mg²⁺ ACSF recordings and before and during application of the drugs.

FIG. 15A is a graph showing the average seizing time (in secs) in rats treated with placebo (vehicle), Phenobarbital only (15 mg/kg), Bumetanide only (0.15 mg/kg) and a combination of Bumetanide (0.15 mg/kg) and Phenobarbital (15 mg/kg).

FIG. 15B is a graph showing the average seizing time comparison between Bumetanide and the control.

### DETAILED DESCRIPTION OF THE INVENTION

NKCCl is a Na⁺—K⁺—Cl⁻ cotransporter expressed in neurons during early development that is thought to mediate the inward chloride ion (Cl⁻) cotransport responsible for intracellular chloride ions (Clᵢ) accumulation, and therefore excitatory GABA responses in neonatal neurons. GABA, the main inhibitory neurotransmitter in the adult brain, normally hyperpolarizes neurons by gating a net influx of anions. However, GABA depolarizes and excites neurons during development, after trauma, in human and experimental epilepsy, in models of neuropathic pain, in normal adult primary sensory neurons, and as a long-term consequence of certain patterns of neuronal activity. In these situations, it is believed that neurons accumulate intracellular chloride ions (Clᵢ) beyond electrochemical equilibrium so that chloride ion (Cl⁻) reversal potential (E_{Cl}) is positive to resting membrane potential, and GABA_{A} receptor activation gates a depolarizing efflux of anions.

With the common incidence of seizures, healthcare providers are in need of innovative treatments to decrease their frequency and intensity. If seizure onset could be effectively modulated, then consequences such as permanent motor or brain insult could be avoided.

One aspect of the invention provides methods for treating a sodium potassium chloride cotransport mediated disorder in a subject. Methods of the present invention are suitable for, but not limited to, treating a neonatal seizure disorder and a chronic epilepsy disorder as well as other sodium potassium chloride cotransport mediated disorders. For the sake of clarity and brevity, the invention is described primarily with reference to neonatal seizures. However, it should be appreciated by those skilled in the art that the embodiments described herein could be easily applied to other types of sodium potassium chloride cotransport mediated disorders, such as seizures. In addition, although the invention is described with reference to human subjects, it should be appreciated that the invention is not limited to human subjects but can be applied to other mammals.

In one embodiment, the invention provides a method for treating neonatal seizure. Typically, methods of the invention include administering a therapeutically effective amount of a composition comprising a diuretic compound. In many instances, methods of the invention comprises administering a pharmaceutical composition, such as chloride ion uptake modulator, to a subject having a sodium/potassium/chloride cotransporter mediated disorder.

The term “modulation” refers to a change in the level or magnitude of an activity or process. The change can be either an increase or a decrease. For example, modulation of γ-aminobutyric acid A (GABA_{A}) receptor activity includes both increase and decrease in GABA_{A} receptor activity. Modulation can be assayed by determining any parameter that is indirectly or directly affecting GABA_{A} receptor activity. Such parameters include, but are not limited to, chloride ion flux.

The term “pharmaceutical composition” refers to one or more agents combined for use in a therapeutic pharmaceutical application to a subject that is acceptable for human use as well as veterinary pharmaceutical use.

“A therapeutically effective amount” means the amount of a compound that, when administered to a subject for treating a disorder, is sufficient to effect such treatment for the disorder. The “therapeutically effective amount” can and will most likely vary depending on the compound, the disorder and its severity and the age, weight, etc., of the subject to be treated.

The term “treating” or “treatment” of a disorder includes: (1) preventing the disorder, i.e., causing the clinical symptoms of the disorder not to develop in a subject; (2) inhibiting the disorder, i.e., arresting or reducing the development of the disorder or its clinical symptoms; or (3) relieving the disorder, i.e., causing regression of the disorder or its clinical symptoms.

As used herein, the term “subject” or “subjects” can include, but are not limited, to humans, birds, reptiles and mammals such as domestic mammals, such as dogs, cats, ferrets, rabbits, pigs, horses, and cattle.

As used herein, “a” or “an” can mean one or more than one of an item.

A composition comprising a chloride uptake modulator, for example, a diuretic compound, refers to a composition, or a pharmaceutically acceptable composition thereof that modulates a chloride ion transport system, such as a sodium/potassium/chloride cotransport system. There are a variety of in vitro assay methods available to determine whether a compound modulates a sodium/potassium/chloride cotransport system. Any known chloride ion uptake modulating compound, such as a diuretic compound, can be used in the methods of the invention. In particular, a chloride ion uptake modulating compound refers to a compound that affects the transport of ions across a membrane, such as a cell membrane, in particular a neuronal cell membrane.

The ion transport affected by the composition of the invention can be sodium, potassium or chloride ion transport or a combination thereof. In one particular embodiment, the sodium/potassium/chloride cotransport system modulated by the composition of the invention includes the NKCCl cotransporter (also referred to as BSC2, bumetanide-sensitive sodium/potassium/chloride cotransporter) or an equivalent cotransporter in a subject. In some embodiments, the chloride ion uptake modulating compound is a diuretic compound. Within these embodiments, in some instances the chloride ion uptake modulating compound is a loop diuretic compound. Exemplary loop diuretic compounds that are suitable for the methods of the invention include, but are not limited to, bumetanide (3-(aminosulfonyl)-5-(butylamino)-4-phenoxybenzoic acid), furosemide (5-(aminosulfonyl)-4-chloro-2-[(2-furanylmethyl)amino] benzoic acid), ethacrynic acid ([2,3-dichloro-4-(2-methylene-1-oxobutyl) phenoxy]acetic acid), and torseamide. In some particular embodiments, the loop diuretic is bumetanide.

In other embodiments, methods of the invention comprise administering a compound capable of decreasing neuronal chloride accumulation in a subject, or administering a pharmaceutical composition comprising such a compound.

In one embodiment, the composition comprises the diuretic compound bumetanide. Bumetanide is a sulfonamide loop diuretic that became clinically available in the 1970s. Loop diuretics block the sodium/potassium/chloride co-transporter in the apical membrane of the thick ascending limb of Henle's loop and the sodium/potassium/chloride co-transporter of neuronal cells. Bumetanide is available from Baxter and Abbott Labs. It is conventionally administered orally, parenterally, or by inhalation. Bumetanide inhibits a sodium/potassium/chloride cotransporter, NKCCl (or BSC2), a transmembrane chloride ion transporter that accumulates chloride ions in neurons. Other loop diuretics include furosemide and ethacrynic acid.

Methods of the invention can be used to treat sodium/potassium/chloride cotransport mediated disorders in infants, children and adult subjects as well as other mammals.

A pharmaceutical composition of the invention can also comprise a second therapeutically useful agent such as a γ-aminobutyric acid A (GABA_{A}) receptor modulator (e.g. an anticonvulsant GABA_{A} receptor modulator, such as tiagabine, acetazolamide, or a combination thereof, and a GABA_{A} receptor positive allosteric modulator, such as a barbiturate, a benzodiazepine, or a combination thereof), an anticonvulsant agent, an antidiuretic agent (e.g., a peripherally-acting antidiuretic agent), or a combination thereof. The term “anticonvulsant agent” refers to an agent that will suppress ictal and/or interictal abnormalities in the EEG activity and behavior.

In other embodiments of the invention, the composition of the invention comprises a compound capable of decreasing neuronal chloride accumulation and a compound capable of modulating GABA production or modulating GABA_{A} receptor activity or a combination thereof.

Formulations

Pharmaceutical compositions as described herein can be administered to a subject to achieve a desired physiological effect. Typically the subject is an animal, more often a mammal, and most typically a human. The pharmaceutical composition can be administered in a variety of forms adapted to the chosen route of administration, e.g., orally, parenterally, or inhalation. Parenteral administration in this respect includes, but are not limited to, administration by the following routes: intravenous; intramuscular; subcutaneous; intraocular; intrasynovial; transepithelially including transdermal, ophthalmic, sublingual and buccal; topically including ophthalmic, dermal, ocular, rectal and nasal inhalation via insufflation and aerosol; intraperitoneal; and rectal systemic.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It can be stable under the conditions of manufacture and storage and must be preserved against the contaminating action of microorganisms such as bacterial and fungi. The carrier can be a solvent of dispersion medium containing, for example, water, ethanol, polyol (e.g., glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, suitable injectable form of the composition includes isotonic agents, e.g., sugars or sodium chloride. Prolonged absorption of the injectable compositions can include agents delaying absorption, e.g., aluminum monostearate and gelatin.

Sterile injectable solutions are prepared by incorporating a pharmaceutical composition of the invention in the required amount in the appropriate solvent with various other ingredients enumerated above, as required, followed by filter sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique which yield a powder of the active ingredient plus any additional desired ingredient from previously sterile-filtered solution thereof.

The pharmaceutical compositions of the invention can be orally administered, for example, with an inert diluent or with an assimilable edible carrier, or it can be enclosed in hard or soft shell gelatin capsules, or it can be compressed into tablets, or it can be incorporated directly with the food of the diet, for example infant formula. For oral therapeutic administration, the pharmaceutical composition can be incorporated with excipient and used in the form of ingestible tablets, buccal tablets, troches, capsules, elixirs, suspensions, syrups, wafers, and the like. Such compositions and preparation can contain at least 0.1% of the pharmaceutical composition of the invention. The percentage of the compositions and preparation can be varied and can conveniently be between about 1 to about 10% of the weight of the unit. The therapeutically useful amount of a pharmaceutical composition is such that a suitable dosage will be obtained. Typical compositions or preparations according to the invention are prepared such that an oral dosage unit form contains from about 1 to about 1000 mg of the pharmaceutical composition herein. In one particular embodiment, the pharmaceutical composition is administered parenterally or by an aerosol delivery system.

The tablets, troches, pills, capsules and the like can also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin can be added or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar or both. A syrup or elixir can contain any of the contemplated pharmaceutical compositions of the invention, sucrose as a sweetening agent, methyl and propylparabens a preservatives, a dye and flavoring such as cherry or orange flavor. Of course, any material used in preparing any dosage unit form should be pharmaceutically pure and substantially non-toxic in the amounts employed. In addition, the pharmaceutical composition can be incorporated into sustained-release preparations and formulation.

A pharmaceutical composition of the invention can be administered to a subject alone or in combination with pharmaceutically acceptable carriers, as noted above, the proportion of which is determined by the solubility and chemical nature of a pharmaceutical composition of the present invention, chosen route of administration and standard pharmaceutical practice.

A physician will determine the dosage of the pharmaceutical composition which will be most suitable for prophylaxis or treatment and it will vary with the form of administration and the particular pharmaceutical composition chosen, and also, it will vary with the particular patient under treatment. The physician will generally wish to initiate treatment with low dosages and by small increments until the optimum effect under the circumstances is reached. The therapeutic dosage can generally be from about 0.1 to about 1000 mg/dose, and preferably from about 0.1 to about 20 mg/Kg of body weight per dose and preferably from about 0.1 to about 50 mg/Kg/of body weight per dose and can be administered in several different dosage units. Higher dosages, on the order of about 2× to about 4×, may be required for oral administration.

In some embodiments, compounds (or the compositions) of the invention are administered through an inhalation route. Accordingly, in some instances compositions of the invention relates to aerosols containing one or more compounds of the invention that are used in inhalation therapy.

In some composition aspects of the invention, the aerosol comprises particles comprising at least 5 percent by weight of an chloride ion uptake modulating compound. Typically, the particles comprise at least 10 percent by weight of a chloride ion uptake modulating compound. More typically, the particles comprise at least 20 percent, 30 percent, 40 percent, 50 percent, 60 percent, 70 percent, 80 percent, 90 percent, 95 percent, 97 percent, 99 percent, 99.5 percent or 99.97 percent by weight of a chloride ion uptake modulating compound.

Typically, the aerosol has a mass of at least 10 μg. More typically, the aerosol has a mass of at least 100 μg. Often, the aerosol has a mass of at least 200 μg.

In other embodiments, the particles comprise less than 10 percent by weight of chloride ion uptake modulating compound degradation products. Often, the particles comprise less than 5 percent by weight of chloride ion uptake modulating compound degradation products. More often, the particles comprise less than 2.5, 1, 0.5, 0.1 or 0.03 percent by weight of chloride ion uptake modulating compound degradation products.

Still in other embodiments, the aerosol particles comprise less than 90 percent by weight of water. Often, the particles comprise less than 80 percent by weight of water. More often, the particles comprise less than 70 percent, 60 percent, 50 percent, 40 percent, 30 percent, 20 percent, 10 percent, or 5 percent by weight of water.

Yet in other embodiments, at least 50 percent by weight of the aerosol is amorphous in form, wherein crystalline forms make up less than 50 percent by weight of the total aerosol weight, regardless of the nature of individual particles. Often, at least 75 percent by weight of the aerosol is amorphous in form. More often, at least 90 percent by weight of the aerosol is amorphous in form.

In other embodiments, the aerosol has an inhalable aerosol particle density of at least 10⁶ particles/mL. Often, the aerosol has an inhalable aerosol particle density of at least 10⁷ particles/mL, more often at least 10⁸ particles/mL.

Still in other embodiments, the aerosol particles have a mass median aerodynamic diameter of 5 microns or less. Often, the particles have a mass median aerodynamic diameter of 3 microns or less. More often, the particles have a mass median aerodynamic diameter of 2 or 1 micron(s) or less.

Yet in other embodiments, the geometric standard deviation around the mass median aerodynamic diameter of the aerosol particles is 3.5 or less. Often, the geometric standard deviation is 3.0 or less. More often, the geometric standard deviation is 2.5 or 2.2 or less.

The aerosol can be formed by any of the methods known to one skilled in the art, such as by heating a composition containing a chloride ion uptake modulating compound to form a vapor and subsequently allowing the vapor to condense into an aerosol or dispersing a fine particles of chloride ion uptake modulating compound in a dispersant and forming an aerosol mist.

In some composition aspects of the invention, the aerosol comprises particles comprising at least 5 percent by weight of 3-(aminosulfonyl)-5-(butylamino)-4-phenoxybenzoic acid, 5-(aminosulfonyl)-4-chloro-2-[(2-furanylmethyl)-amino]benzoic acid, [2,3-dichloro-4-(2-methylene-1-oxobutyl) phenoxy]acetic acid, or a combination thereof. Often, the particles comprise at least 10 percent by weight of 3-(aminosulfonyl)-5-(butylamino)-4-phenoxybenzoic acid, 5-(aminosulfonyl)-4-chloro-2-[(2-furanylmethyl)-amino]benzoic acid, [2,3-dichloro-4-(2-methylene-1-oxobutyl) phenoxy]acetic acid, or a combination thereof. More often, the particles comprise at least 20 percent, 30 percent, 40 percent, 50 percent, 60 percent, 70 percent, 80 percent, 90 percent, 95 percent, 97 percent, 99 percent, 99.5 percent or 99.97 percent by weight of 3-(aminosulfonyl)-5-(butylamino)-4-phenoxybenzoic acid, 5-(aminosulfonyl)-4-chloro-2-[(2-furanylmethyl)-amino]benzoic acid, [2,3-dichloro-4-(2-methylene-1-oxobutyl) phenoxy]acetic acid, or a combination thereof.

Typically, the aerosol has a mass of at least 10 μg. Often, the aerosol has a mass of at least 100 μg. More often, the aerosol has a mass of at least 200 μg.

Typically, the aerosol particles comprise less than 10 percent by weight of 3-(aminosulfonyl)-5-(butylamino)-4-phenoxybenzoic acid, 5-(aminosulfonyl)-4-chloro-2-[(2-furanylmethyl)-amino]benzoic acid, [2,3-dichloro-4-(2-methylene-1-oxobutyl) phenoxy]acetic acid, or a combination thereof degradation products. Often, the particles comprise less than 5 percent by weight of 3-(aminosulfonyl)-5-(butylamino)-4-phenoxybenzoic acid, 5-(aminosulfonyl)-4-chloro-2-[(2-furanylmethyl)-amino]benzoic acid, [2,3-dichloro-4-(2-methylene-1-oxobutyl) phenoxy]acetic acid, or a combination thereof degradation products. More often, the particles comprise less than 2.5, 1, 0.5, 0.1 or 0.03 percent by weight of 3-(aminosulfonyl)-5-(butylamino)-4-phenoxybenzoic acid, 5-(aminosulfonyl)-4-chloro-2-[(2-furanylmethyl)-amino]benzoic acid, [2,3-dichloro-4-(2-methylene-1-oxobutyl) phenoxy]acetic acid, or a combination thereof degradation products.

Typically, the aerosol has an inhalable aerosol drug mass density of about 5 mg/L or more. Often, the aerosol has an inhalable aerosol drug mass density of about 7.5 mg/L or more. More often, the aerosol has an inhalable aerosol drug mass density of about 10 mg/L or more.

The aerosol can be formed by any methods known to one skilled in the art including by heating a composition containing 3-(aminosulfonyl)-5-(butylamino)-4-phenoxybenzoic acid, 5-(aminosulfonyl)-4-chloro-2-[(2-furanylmethyl)-amino]benzoic acid, [2,3-dichloro-4-(2-methylene-1-oxobutyl) phenoxy]acetic acid, or a combination thereof to form a vapor and subsequently allowing the vapor to condense into an aerosol.

In some aspects of the invention, an NSAID is delivered to a mammal through an inhalation route. The method comprises forming a vapor particle of a chloride ion uptake modulating compound; and allowing the vapor to cool, thereby forming a condensation aerosol comprising particles. Such condensed aerosol particles can be inhaled by the mammal or can be formulated to be inhaled by the mammal.

Typically, the rate of inhalable aerosol particle formation of the delivered condensation aerosol is 10⁸ particles per second or greater. Often, the aerosol is formed at a rate 10⁹ inhalable particles per second or greater. More often, the aerosol is formed at a rate 10¹⁰ inhalable particles per second or greater.

Typically, the delivered condensation aerosol is formed at a rate 0.5 mg/second or greater. Often, the aerosol is formed at a rate 0.75 mg/second or greater. More often, the aerosol is formed at a rate 1 mg/second, 1.5 mg/second or 2 mg/second or greater.

Typically, the delivered condensation aerosol results in a peak plasma concentration of a chloride ion uptake modulating compound in the mammal in 1 h or less. Often, the peak plasma concentration is reached in 0.5 h or less. More often, the peak plasma concentration is reached in 0.2, 0.1, 0.05, 0.02, 0.01, or 0.005 h or less (arterial measurement).

“Aerodynamic diameter” of a given particle refers to the diameter of a spherical droplet with a density of 1 g/mL (the density of water) that has the same settling velocity as the given particle.

“Aerosol” refers to a suspension of solid or liquid particles in a gas.

“Aerosol drug mass density” refers to the mass of a compound of interest per unit volume of aerosol.

“Aerosol mass density” refers to the mass of particulate matter per unit volume of aerosol.

“Aerosol particle density” refers to the number of particles per unit volume of aerosol.

“Amorphous particle” refers to a particle that contains about 50 percent by weight or less of a crystalline form. Often, the particle contains about 25 percent by weight or less of a crystalline form. More often, the particle contains about 10 percent by weight or less of a crystalline form.

“Condensation aerosol” refers to an aerosol formed by vaporization of a substance followed by condensation of the substance into an aerosol.

“Inhalable aerosol drug mass density” refers to the aerosol drug mass density produced by an inhalation device and delivered into a typical patient tidal volume.

“Inhalable aerosol mass density” refers to the aerosol mass density produced by an inhalation device and delivered into a typical patient tidal volume.

“Inhalable aerosol particle density” refers to the aerosol particle density of particles of size between 100 nm and 5 microns produced by an inhalation device and delivered into a typical patient tidal volume.

“Mass median aerodynamic diameter” or “MMAD” of an aerosol refers to the aerodynamic diameter for which half the particulate mass of the aerosol is contributed by particles with an aerodynamic diameter larger than the MMAD and half by particles with an aerodynamic diameter smaller than the MMAD.

“Rate of aerosol formation” refers to the mass of aerosolized particulate matter produced by an inhalation device per unit time.

“Rate of inhalable aerosol particle formation” refers to the number of particles of size between 100 nm and 5 microns produced by an inhalation device per unit time.

“Rate of drug aerosol formation” refers to the mass of aerosolized chloride ion uptake modulator compound produced by an inhalation device per unit time.

Any suitable method is used to form the aerosols of the present invention. One particular method involves heating a composition comprising a chloride ion uptake modulating compound to form a vapor, followed by cooling of the vapor such that it condenses to provide a chloride ion uptake modulating compound comprising aerosol (condensation aerosol). The composition can be heated in one of four forms: as pure active compound; as a mixture of active compound and a pharmaceutically acceptable excipient; as a salt form of the pure active compound; and, as a mixture of active compound salt form and a pharmaceutically acceptable excipient.

Salt forms of chloride ion uptake modulating compounds are either commercially available or are obtained from the corresponding free base using well known methods in the art. A variety of pharmaceutically acceptable salts are suitable for aerosolization. Such salts include, without limitation, the following: hydrochloric acid, hydrobromic acid, acetic acid, maleic acid, formic acid, and fumaric acid salts.

Pharmaceutically acceptable excipients can be volatile or nonvolatile. Volatile excipients, when heated, are concurrently volatilized, aerosolized and inhaled with the chloride ion uptake modulating compound. Classes of such excipients are known in the art and include, without limitation, gaseous, supercritical fluid, liquid and solid solvents. The following is a list of exemplary carriers within the classes: water; terpenes, such as menthol; alcohols, such as ethanol, propylene glycol, glycerol and other similar alcohols; dimethylformamide; dimethylacetamide; wax; supercritical carbon dioxide; dry ice; and mixtures thereof.

Solid supports on which the composition is heated are of a variety of shapes. Examples of such shapes include, without limitation, cylinders of about 1 mm or less in diameter, boxes of about 1 mm thickness or less and virtually any shape permeated by small (e.g., about 1 mm-sized or less) pores. Typically, solid supports provide a large surface to volume ratio (e.g., about 100 per meter or more) and a large surface to mass ratio (e.g., about 1 cm² per gram or more).

A solid support of one shape can also be transformed into another shape with different properties. For example, a flat sheet of 0.25 mm thickness has a surface to volume ratio of approximately 8,000 per meter. Rolling the sheet into a hollow cylinder of 1 cm diameter produces a support that retains the high surface to mass ratio of the original sheet but has a lower surface to volume ratio (about 400 per meter).

A number of different materials are used to construct the solid supports. Classes of such materials include, without limitation, metals, inorganic materials, carbonaceous materials and polymers. The following are examples of the material classes: aluminum, silver, gold, stainless steel, copper and tungsten; silica, glass, silicon and alumina; graphite, porous carbons, carbon yarns and carbon felts; polytetrafluoroethylene and polyethylene glycol. Combinations of materials and coated variants of materials are used as well.

Where aluminum is used as a solid support, aluminum foil is a suitable material. Examples of silica, alumina and silicon based materials include amphorous silica S-5631 (Sigma, St. Louis, Mo.), BCR171 (an alumina of defined surface area greater than 2 m²/g from Aldrich, St. Louis, Mo.) and a silicon wafer as used in the semiconductor industry. Carbon yarns and felts are available from American Kynol, Inc., New York, N.Y. Chromatography resins such as octadecycl silane chemically bonded to porous silica are exemplary coated variants of silica.

The heating of the chloride ion uptake modulating compositions is performed using any suitable method. Examples of methods by which heat can be generated include the following: passage of current through an electrical resistance element; absorption of electromagnetic radiation, such as microwave or laser light; and, exothermic chemical reactions, such as exothermic salvation, hydration of pyrophoric materials and oxidation of combustible materials.

Other methods for generating aerosols are well known to one skilled in the art. For example, aerosol particles can be generated by dispersing fine particles of a composition comprising a chloride ion uptake modulating compound with a dispersant. Suitable dispersants are well known to one skilled in the art.

Dosage of Chloride Ion Uptake Modulating Compound Containing Aerosols

The dosage amount of a chloride ion uptake modulating compound (or a composition comprising such a compound) in aerosol form is generally no greater than twice the standard dose of the drug given orally; oftentimes, the dose is less than the standard oral dose. A typical dosage of a chloride ion uptake modulating compound aerosol is either administered as a single inhalation or as a series of inhalations taken within an hour or less (dosage equals sum of inhaled amounts). Where the drug is administered as a series of inhalations, a different amount can be delivered in each inhalation.

One can determine the appropriate dose of chloride ion uptake modulating compound containing aerosols to treat a particular condition using methods such as animal experiments and a dose-finding (Phase I/II) clinical trial. One animal experiment involves measuring plasma concentrations of drug in an animal after its exposure to the aerosol. Mammals such as dogs or primates are typically used in such studies, since their respiratory systems are similar to that of a human. Initial dose levels for testing in humans is generally less than or equal to the dose in the mammal model that resulted in plasma drug levels associated with a therapeutic effect in humans. Dose escalation in humans is then performed, until either an optimal therapeutic response is obtained or a dose-limiting toxicity is encountered.

Analysis of NSAID Containing Aerosols

Purity of a chloride ion uptake modulating compound containing aerosol is determined using a number of methods, examples of which are described in Sekine et al., Journal of Forensic Science, 1987, 32, 1271-1280 and Martin et al., Journal of Analytic Toxicology, 1989, 13, 158-162. One method involves forming the aerosol in a device through which a gas flow (e.g., air flow) is maintained, generally at a rate between 0.4 and 60 L/min. The gas flow carries the aerosol into one or more traps. After isolation from the trap, the aerosol is subjected to an analytical technique, such as gas or liquid chromatography, that permits a determination of composition purity.

A variety of different traps are used for aerosol collection. The following list contains examples of such traps: filters; glass wool; impingers; solvent traps, such as dry ice-cooled ethanol, methanol, acetone and dichloromethane traps at various pH values; syringes that sample the aerosol; empty, low-pressure (e.g., vacuum) containers into which the aerosol is drawn; and, empty containers that fully surround and enclose the aerosol generating device. Where a solid such as glass wool is used, it is typically extracted with a solvent such as ethanol. The solvent extract is subjected to analysis rather than the solid (i.e., glass wool) itself Where a syringe or container is used, the container is similarly extracted with a solvent.

The gas or liquid chromatograph discussed above contains a detection system (i.e., detector). Such detection systems are well known in the art and include, for example, flame ionization, photon absorption and mass spectrometry detectors. An advantage of a mass spectrometry detector is that it can be used to determine the structure of chloride ion uptake modulating compound degradation products.

Particle size distribution of a chloride ion uptake modulating compound containing aerosol is determined using any suitable method in the art (e.g., cascade impaction). An Andersen Eight Stage Non-viable Cascade Impactor (Andersen Instruments, Smyrna, Ga.) linked to a furnace tube by a mock throat (USP throat, Andersen Instruments, Smyrna, Ga.) is one system used for cascade impaction studies.

Inhalable aerosol mass density is determined, for example, by delivering a drug-containing aerosol into a confined chamber via an inhalation device and measuring the mass collected in the chamber. Typically, the aerosol is drawn into the chamber by having a pressure gradient between the device and the chamber, wherein the chamber is at lower pressure than the device. The volume of the chamber should approximate the tidal volume of an inhaling patient.

Inhalable aerosol drug mass density is determined, for example, by delivering a drug-containing aerosol into a confined chamber via an inhalation device and measuring the amount of active drug compound collected in the chamber. Typically, the aerosol is drawn into the chamber by having a pressure gradient between the device and the chamber, wherein the chamber is at lower pressure than the device. The volume of the chamber should approximate the tidal volume of an inhaling patient. The amount of active drug compound collected in the chamber is determined by extracting the chamber, conducting chromatographic analysis of the extract and comparing the results of the chromatographic analysis to those of a standard containing known amounts of drug.

Inhalable aerosol particle density is determined, for example, by delivering aerosol phase drug into a confined chamber via an inhalation device and measuring the number of particles of given size collected in the chamber. The number of particles of a given size may be directly measured based on the light-scattering properties of the particles. Alternatively, the number of particles of a given size is determined by measuring the mass of particles within the given size range and calculating the number of particles based on the mass as follows: Total number of particles=Sum (from size range 1 to size range N) of number of particles in each size range. Number of particles in a given size range=Mass in the size range/Mass of a typical particle in the size range. Mass of a typical particle in a given size range=π*D³*φ/6, where D is a typical particle diameter in the size range (generally, the mean boundary MMADs defining the size range) in microns, φ the particle density (in g/mL) and mass is given in units of picograms (g⁻¹²).

Rate of inhalable aerosol particle formation is determined, for example, by delivering aerosol phase drug into a confined chamber via an inhalation device. The delivery is for a set period of time (e.g., 3 s), and the number of particles of a given size collected in the chamber is determined as outlined above. The rate of particle formation is equal to the number of 100 nm to 5 micron particles collected divided by the duration of the collection time.

Rate of aerosol formation is determined, for example, by delivering aerosol phase drug into a confined chamber via an inhalation device. The delivery is for a set period of time (e.g., 3 s), and the mass of particulate matter collected is determined by weighing the confined chamber before and after the delivery of the particulate matter. The rate of aerosol formation is equal to the increase in mass in the chamber divided by the duration of the collection time. Alternatively, where a change in mass of the delivery device or component thereof can only occur through release of the aerosol phase particulate matter, the mass of particulate matter may be equated with the mass lost from the device or component during the delivery of the aerosol. In this case, the rate of aerosol formation is equal to the decrease in mass of the device or component during the delivery event divided by the duration of the delivery event.

Rate of drug aerosol formation is determined, for example, by delivering a chloride ion uptake modulating compound containing aerosol into a confined chamber via an inhalation device over a set period of time (e.g., 3 s). Where the aerosol is pure chloride ion uptake modulating compound, the amount of drug collected in the chamber is measured as described above. The rate of drug aerosol formation is equal to the amount of chloride ion uptake modulating compound collected in the chamber divided by the duration of the collection time. Where the chloride ion uptake modulating compound containing aerosol comprises a pharmaceutically acceptable excipient, multiplying the rate of aerosol formation by the percentage of chloride ion uptake modulating compound in the aerosol provides the rate of drug aerosol formation.

Utility Of Chloride Ion Uptake Modulating Compound Containing Aerosols

The chloride ion uptake modulating compound containing aerosols of the present invention are typically used for the treatment of NKCCl mediated disorders. With aerosol delivery, it is believed the chloride ion uptake modulating compound can be delivered to the brain via the olfactory bulb and does not have to cross the blood-brain barrier. Accordingly, a smaller amount of drug may be required in an aerosol form. Moreover, aerosols of the chloride ion uptake modulating compound may be more efficacious, and may be faster acting relative to other means of delivery.

Pharmaceutical Composition

The pharmaceutical compositions of the invention have a variety of physiological properties including modulating, typically reducing or inhibiting, ion transport activity for example chloride ion uptake. In particular, some pharmaceutical compositions of the invention are found to be antagonists of ion transporters. Other pharmaceutical compositions of the invention are found to be agonists of ion transporters. Therefore, they can be used in a variety of applications where modulating ion transport activity in a subject is desired. For example, pharmaceutical compositions of the invention can be used to treat a sodium/potassium/chloride cotransport mediated disorder in a subject. Exemplary disorders that are mediated by sodium/potassium/chloride cotransport include, but are not limited to, neonatal seizure disorders and chronic epilepsy disorders.

Additional objects, advantages, and novel features of the invention will become apparent to those skilled in the art upon examination of the following examples thereof, which are not intended to be limiting. Procedures that are constructively reduced to practice are described in the present tense, and procedures that have been carried out in the laboratory are set forth in the past tense.

### EXAMPLES

The accompanying drawings form part of the specification and are included to further demonstrate certain aspects of the invention. The invention may be better understood by reference to one or more of these drawings in combination with the detailed description of specific embodiments presented herein.

Sodium Potassium Chloride Cotransport Mediated Disorders

Seizures

Neonatal seizures are potentially catastrophic conditions affecting children in the first month of life. Their appearances may lead to severe neurological dysfunction and their presence may be powerful predictors of long-term cognitive impairment and reduced seizure threshold. Neonatal seizures are difficult to treat, and even when the clinical manifestations are suppressed by treatment, EEG recordings demonstrate ongoing cortical seizure activity. Severe side effects of seizures can include cerebral palsy, hydrocephalus, epilepsy, spasticity and/or feeding problems.

Seizures occur when a large group of neurons undergo excessive, synchronized depolarization. Depolarization can result from excessive excitatory amino acid release (e.g. glutamate) or deficient inhibitory neurotransmitter (e.g. GABA). Another potential cause is disruption of ATP-dependent resting membrane potentials, which causes a flow of sodium into the neuron and potassium out of the neuron. Hypoxic-ischemic encephalopathy disrupts the ATP-dependent sodium-potassium pump and appears to cause excessive depolarization.

Neonatal seizures by definition occur within the first month of life in a full-term infant and up to 44 weeks from conception for premature infants. Seizures are most frequent during the first 10 days of life. Seizures are due to a large variety of conditions. It may be difficult to determine if a newborn is actually seizing, since they often do not have convulsions. Instead, a newborn's eyes appear to be looking in different directions. Sometimes they may have lip smacking or periods of no breathing.

Most seizures in the neonate are focal, although generalized seizures have been described in rare instances. Subtle seizures are more common in full-term than in premature infants. Video EEG studies have demonstrated that most subtle seizures are not associated with electrographic seizures. Examples of subtle seizures include chewing, pedaling, or ocular movements.

Clonic seizures are associated with electrographic seizures. They often involve one extremity or one side of the body. The rhythm of the clonic movements is usually slow, 1-3 movements per second.

Tonic seizures can involve one extremity or the whole body. Focal tonic seizures involving one extremity often are associated with electrographic seizures. Generalized tonic seizures often manifest with tonic extension of both upper and lower limbs and also may involve the axial musculature.

Generalized tonic seizures mimic decorticate posturing; the majority are not associated with electrographic seizures. For example, myoclonic seizures may occur focally in one extremity or in several body parts (in which case they are described as multifocal myoclonic seizures). Focal and multifocal myoclonic seizures typically are not associated with electrographic correlates.

The differential causes for neonatal seizures is a large category. In one example, seizures can result from hypoxic-ischemic encephalopathy seen in both term and premature infants. A seizure frequently presents within the first 72 hours of life. Seizures may include subtle, clonic, or generalized seizures. Intracranial hemorrhage occurs more frequently in premature than in term infants.

Subarachnoid hemorrhage is more common in term infants but, infants with subarachnoid hemorrhage appear remarkably well. But, germinal matrix-intraventricular hemorrhage is seen more frequently in premature than in term infants, particularly in infants born prior to 34 weeks' gestation. Subtle seizures are seen frequently with this type of hemorrhage. Subdural hemorrhage is seen in association with cerebral contusion. It is more common in term infants.

Metabolic disturbances may also trigger seizures and these disturbances include hypoglycemia, hypocalcemia, and hypomagnesemia. Less frequent metabolic disorders, such as inborn errors of metabolism, are seen more commonly in infants who are older than 72 hours. Typically, they may be seen after the infant starts feeding.

Intracranial infections such as meningitis, encephalitis (including herpes encephalitis), toxoplasmosis, and cytomegalovirus (CMV) infections can cause neonatal seizures. The common bacterial pathogens that can cause a seizure include Escherichia coli and Streptococcus pneumoniae.

Other Disorders

Other disorders can be treated by a pharmaceutical composition of the present invention include, but are not limited to kidney disorders, seizure disorders in children, seizure disorders in adults and seizure disorders in non-humans for example domestic animals.

GABA and GABA_{A} and Seizures

In the developing hippocampus activation of anion permeable GABA receptors excites many neurons, and this has been linked to a higher seizure propensity. The excitatory action of GABA depends on elevated intracellular chloride ion levels and a depolarized chloride ion equilibrium potential. In a more mature subject inhibitory function of GABA is acquired as net outward neuronal chloride transport develops in a caudal-rostral progression. In line with this caudal-rostral developmental pattern, GABA stimulating anticonvulsants inhibit motor manifestations of neonatal seizures but not cortical seizure activity.

Neonatal seizures and intractable temporal lobe epilepsy (TLE) are two disorders that anticonvulsants are not sufficiently effective. Both disorders share a common feature: GABA, which becomes excitatory due to neuronal accumulation of intracellular chloride. GABA normally inhibits neurons by gating the net influx of anions. However, during development, following trauma, in neuropathic pain models, in human and experimental epilepsy, and as a long-term consequence of certain patterns of synaptic activity, neurons accumulate intracellular chloride beyond electrochemical equilibrium. Under these conditions, GABA_{A} receptor activation is excitatory due to net efflux of anions.

One cause of neuronal accumulation of intracellular chloride is linked to a cell surface membrane-associated electroneutral sodium/potassium/chloride cotransport system expressed in most mammalian cells (rat NKCCl or human BSC2). One function of this system is vectorial transport of ions across certain polarized epithelia. Specifically, in vascular endothelium, this system contributes to the regulation of a selective permeability barrier at the blood-tissue interface in certain organs (e.g., the “blood-brain” barrier in the central nervous system), as well as the integrity of the vascular lining in response to fluctuations in ambient osmotic conditions. Because of the importance of electroneutral sodium/potassium/chloride cotransport in the adaptation of neuronal cells (and other cell types) to varying physiologic and pathophysiologic conditions, the regulation of this class of transporters is critical. In addition, this system is a molecular target of loop diuretics and the human transporter is termed bumetanide-sensitive sodium/potassium/chloride cotransporter (BSC2).

Sodium/Potassium/Chloride Cotransport System

One sodium/potassium/chloride cotransport system (NKCCl or BSC2) encodes a more widely expressed cotransporter. This cotransporter (also known as the secretory form) functions in intracellular volume regulation, as well as more specialized functions such as salt and water secretion across respiratory, sweat gland, and salivary epithelia. It is a member of the large superfamily of membrane-spanning transporters whose members serve diverse functions ranging from the transport of inorganic and organic ions to the regulation of macrophage activation and antimicrobial activity. Neuronal chloride ion concentrations are maintained by 2 transporters: KCC2 (Potassium Chloride cotransporter 2) exports chloride ions out of the cytoplasm, and NKCCl imports chloride ions into the cytoplasm.

Chloride Ions Export Via KCC2 in Mature Neurons

The cytoplasmic chloride ion concentration in mature neurons is maintained at only a few millimolar, while the extracellular chloride ion concentration is 20 times that. Thus, the chloride reversal potential can be dramatically altered by a small change in chloride ions. This can be enough to cause GABA to trigger action potentials with only a small change in intracellular chloride concentration. Therefore, intracellular chloride concentration must be strictly controlled in order to maintain the inhibitory effect of GABA_{A} receptor activation. In the adult, the electroneutral K—Cl cotransporter KCC2 uses the energy stored in the transmembrane K⁺ gradient to cotransport K+ and Cl⁻ out of the cytoplasm.

Chloride follows its electrochemical gradient through the GABA_{A} receptor. KCC2 uses the energy stored in the potassium gradient to export chloride from the cytoplasm. NKCCl uses energy stored in the sodium gradient to import 1 potassium and 2 chloride ions into the cytoplasm. In the adult, KCC2 activity is very active, resulting in low intracellular chloride concentrations, so chloride flow through the GABA_{A} receptor is inward and hyperpolarizing; but in neonatal neurons and in pathological conditions, NKCCl activity is thought to be more active than KCC2, and chloride accumulation in the cytoplasm results in a depolarizing efflux of chloride ion through the GABA_{A} receptor.

Thus, in immature neonatal neurons, chloride is typically maintained several millimolar higher than in the adult where chloride leaves the cytoplasm via open GABA_{A} channels, depolarizing the neuron and triggering action potentials. Thus, immature neurons must accumulate chloride ions beyond equilibrium. This entails limiting chloride ion export via KCC2, which occurs via regulation of KCC2 expression. However, limiting chloride export is not sufficient to make GABA excitatory, because a lack of active export only brings the transmembrane chloride ion gradient to electrochemical equilibrium, e.g. E_{Cl}=RMP (resting membrane potential) and no chloride ion efflux would occur during GABA_{A} receptor activation. Under these conditions GABA_{A} receptor activation would still be inhibitory due to shunting of excitatory synaptic activity.

Neonatal Seizures and Sodium Potassium Chloride Cotransport

Based on the regional differences in KCC2 ontogeny, there is a developmental stage at which GABA inhibits the spinal cord and brainstem neurons, and yet still excites cortical neurons. At this developmental stage, anticonvulsants that prolong the open time of the GABA_{A} receptor, such as barbiturates and benzodiazepines, do not decrease or stop cortical seizure activity, but do inhibit the brainstem and spinal cord activity, which could block the motor manifestations of the seizure. In previous studies, EEGs of human neonatal seizures have demonstrated that the EEG rarely responds to phenobarbital and benzodiazepines, although motor manifestations of seizures are often blocked. Therefore, the human neonate is in a developmental window during which the GABA is inhibitory in the brainstem and spinal cord, but GABA is excitatory in the cortex.

In neonatal rats, where cortical KCC2 expression data confirms the activity of the NKCCl cotransporter by inhibiting chloride accumulation in the hippocampus in vitro or the entire brain in vivo, produces a profound electrographic anticonvulsant effect. Other data indicates that the neonatal human cortex has similarly immature chloride ion transport as the rodent.

Murine Seizure Model

Kainic Acid (KA) is a commonly used agent for creating an in vivo neonatal-like seizure model in rats for study. It is believed that this seizure model closely parallels human neonatal seizure disorders. KA, a glutamate receptor agonist, was used to induce neonatal-like seizures in postnatal (P) 7-12 day male Wistar rats. The rats were equipped with electrodes including recording and reference electrodes by standard means. There are many known methods for measuring parameters of seizures, in this example electrical signals were digitized using an analogue-to-digital converter for example a DigiData 1322A (Axon Instuments USA). The sampling interval can vary depending on the desired target interval. In this example, the sampling interval per signal was 200 □s (5 kHz). For data analysis, several computer programs exist for capturing and analyzing these seizure parameters such as Clamp 9.2 (Axon Instruments, USA) and Origon 5.0 (Microcal Software, USA) used here. Interictal spikes were determined by typical means utilizing band-pass filtered raw data.

Many known methods exist for analyzing the power levels of different frequency components in a signal such as a signal recorded from a seizure episode. For example, power spectrum analysis can be performed after applying a Hamming window function. Unless otherwise indicated, power was calculated in 1-2 min time windows by integrating the root mean square value of the signal in frequency bands from 1 to 100 Hz (EEC band) and from 200 to 500 Hz (fast ripple band).

Effects of Inhibition of a Sodium/Potassium/Chloride Cotransporter (NKCCl)

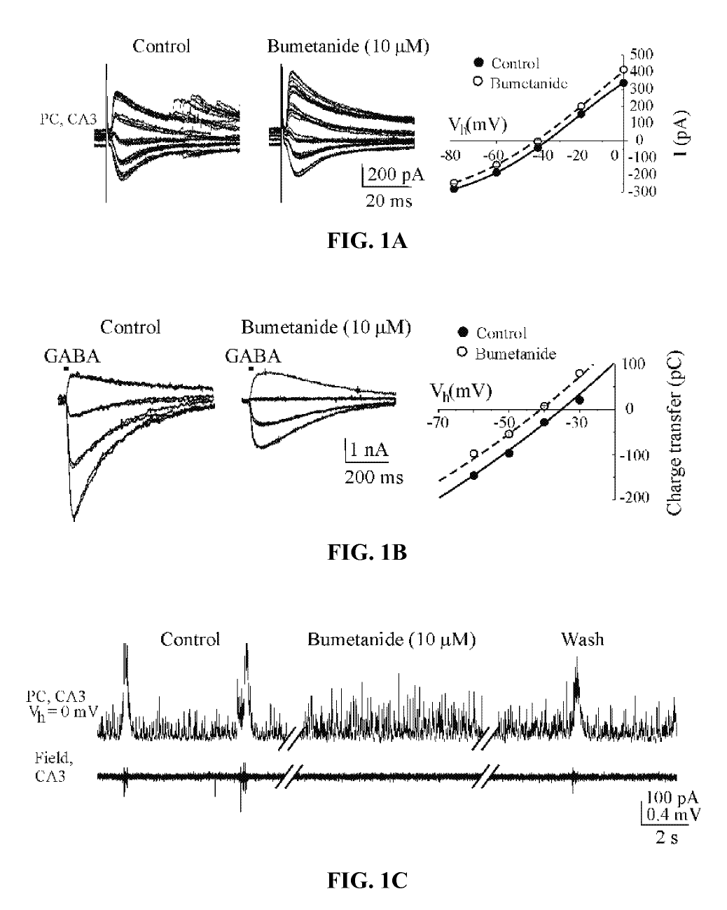

Cell cultures, such as pyramidal cells, can be used to examine the effects of a pharmaceutical composition of the present invention on GABA_{A} receptor activation. In one exemplary method, selective inhibition of NKCCl by a pharmaceutical composition of the present invention, bumetanide, was tested to examine the relationship between chloride accumulation in neurons, GABA_{A} receptor activation and NKCC1. Specifically, the reversal potential of electrical stimulus evoked GABA_{A}-receptor mediated post-synaptic currents (GABA_{A}-PSCS) in immature P4-P6 CA3 pyramidal cells of the hippocampus was examined in the presence or absence of bumetanide. Electrical stimulation of the stratum radiatum (7 V, 30 us duration) evoked in control conditions a synaptic response that reversed at −42.4±1.7 mV (n=6) and was close to the reversal potential of spontaneous GABA_{A}-PSCS (−46.1±0.7 mV; n=6, p=0.07). Examples of stimulus evoked postsynaptic currents at different holding potentials for individual pyramidal cell and corresponded current-voltage (I-V) relationship are shown in FIG. 1A. Pharmacological inhibition of a sodium/potassium/chloride cotransporter by bumetanide (10 □M) application led to a negative shift in reversal potential of GABA_{A}-PSCs. In presence of bumetanide, stimulus evoked GABA_{A}-PSCs reversed at −46.1±1.9 mV (n=6, p=0.17). Thus, the inhibition of NKCCl indicates that the NKCCl is at least one target for controlling chloride ion transport into neuronal cells and GABA_{A} receptor mediated post-synaptic current activation. This in vitro system can be used to test any of the pharmaceutical compositions of the present invention for modulation of GABA_{A} receptor activation.

In one example, direct effect of bumetanide on Egaba (excitatory GABA_{A}) can be tested. Here, whole-cell voltage clamp recordings to measure the reversal potential of currents elicited by local application of GABA (10 pM, 10 ms pulse duration) in CA1 pyramidal cells were used. As shown in FIG. 1B, Egaba (GABA excitation) was more hyperpolarized in the presence of bumetanide (−40.35+2.73 mV, n=6 cells from 8; p=0.005) than in control (−37±2.72 mV, n-8).

In another exemplary in vitro method, simultaneous whole-cell voltage-clamp recordings of CA3 pyramidal cells and extracellular field potential recordings in CA3 pyramidal cell layer were performed to analyze the effects of one of the pharmaceutical compositions of the present invention, bumetanide, on spontaneous network activity in the postnatal (P)4-8 hippocampal sections. Spontaneous neuronal network activity is characterized by large polysynaptic currents mediated by activation of GABA_{A}-receptors and synchronous recurrent population bursts, associated with a barrage of high frequency action potentials from multiple cells (FIG. 1C). In one exemplary method, bath application of bumetanide (10 uM) rapidly depressed synchronous bursts of action potentials (n=10). Synchronous network activity reappeared 10-15 min after removal of bumetanide by washing. Therefore, this investigation reveals that bumetanide has beneficial effects on reducing these spontaneous recurrent population bursts mediated by the GABA_{A} receptor activation.

Anti-Convulsant Effects on Seizures in the Developing Hippocampus

To determine if a pharmaceutical composition of the present invention modulates GABA-induced effects on seizure activity, the composition can be applied to a seizure-induced rat hippocampus section. In one exemplary method, one of the typically used medical treatments for neonatal seizures, phenobarbital was found to increase GABA effects. While these compounds are effective anticonvulsants in the more mature brain, the excitatory effects of GABA in immature neurons renders these anticonvulsants ineffective alone for treatment of neonatal seizures, as is seen upon examination of EEG recordings of phenobarbital and benzodiazepines (data not shown) of this neonatal seizure model.

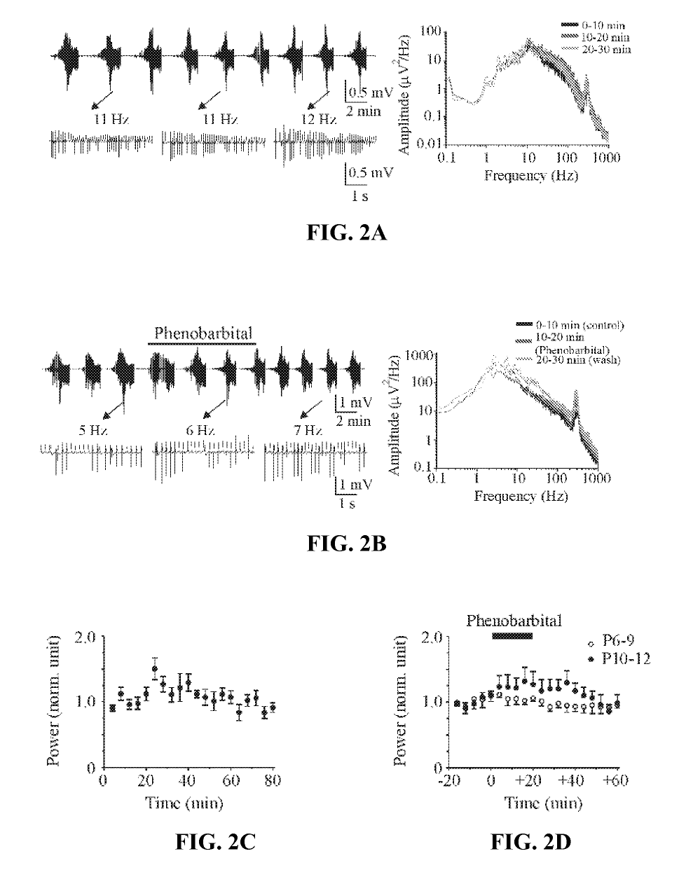

In this example, the effects of phenobarbital on recurrent interictal and ictal-like epileptiform activity in the hippocampal slices in vitro from P7-12 rats were studied. Interictal and ictal-like epileptiform activity are typical parameters studied when analyzing seizure activity in control and treated subjects. Phenobarbital exerts its pharmacological effects by allosteric activation of the GABA_{A} receptor, increasing the duration of chloride ion channel opening, without affecting the frequency of opening or channel conductance. Simultaneous multi-site extracellular field potential records were performed in the CA3 pyramidal cell layer. To induce progressive neuronal firing, a bath application of 8.5 mM K+ was applied to the sections that developed to recurrent interictal and ictal-like epileptiform activities (FIG. 2A-2D). In one example, bath application of phenobarbital (100 □M) did not suppress epileptiform activity in hippocampal slices from P7-9 rats (n=6) and PI0-12 rats (n=7; FIGS. 2B and 2D). The power of extracellular field potential during 8.5 mM K+ (potassium) induced epileptiform activity in 0.1-1000 Hz frequency band, that include both gamma and fast ripple oscillations (FIG. 2A-2D). Averaged power of epileptiform activity in control K+ treated samples and following phenobarbital application was not significantly different (p=0.09 for P7-9 and p+0.06 in P10-12; FIG. 2 A-2D).

NKCCl Transporter and Seizures in the Developing Hippocampus

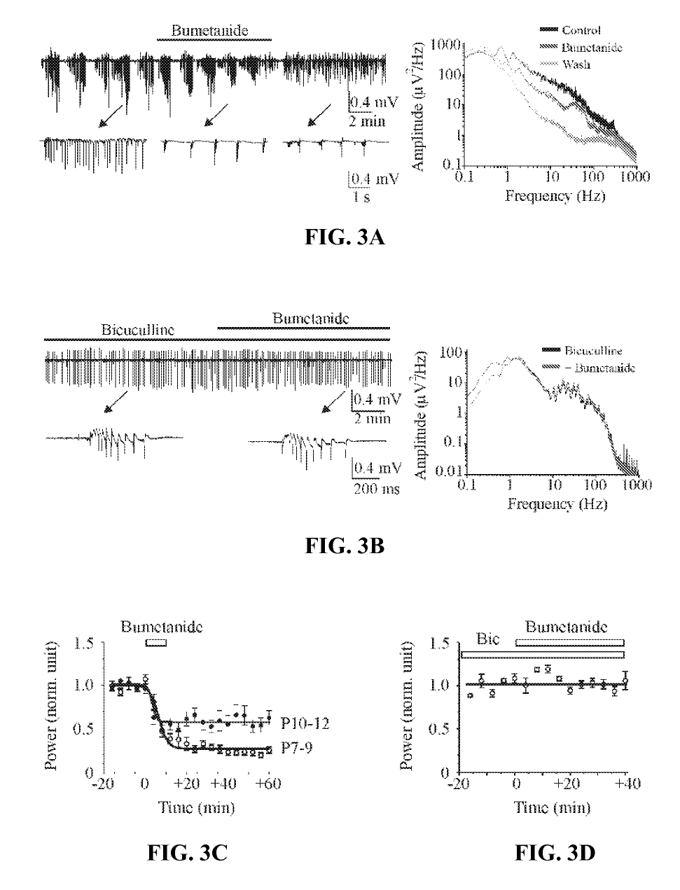

In another exemplary method, the NKCCl transporter, was investigated and found to be a contributing factor to seizures in developing hippocampus samples. During development of inhibitory synapses, the action of the GABA shifts from depolarizing to hyperpolarizing. The shift is due to an age-dependent regulation of the intracellular free chloride concentration in postsynaptic neurons as previously indicated. The NKCCl transporter was tested for activity related to accumulation of intracellular chloride ions, depolarizing action of GABA and higher seizure propensity in the neonatal brain, by inhibiting ion transport activity (FIG. 3A-3D).

In Vitro Assay Method

Pharmaceutical compositions of the present invention such as chloride ion uptake modulators may be tested for modulation of seizures for example in the seizure induced developing hippocampus samples. Alternatively, pharmaceutical compositions of the present invention may be administered to rats to assess the seizure inhibitory capabilities against seizure-induced rat models such as neonatal seizure models. The results of these studies can be used to assess the efficacy of a pharmaceutical composition alone or in combination with other treatments to modulate seizure activity in a subject such as a human subject.

In one exemplary method, experimental procedures were carried out on hippocampal slices of postnatal days 5 (P5) to P12 male Wistar rats. Animals were anaesthetized and decapitated and hippocampal slices were prepared by known methods. For example, hippocampal transverse slices were cut using Leica VT-100E vibratome (Leica Microsystems Nussloch GmbH, Germany) and kept in oxygenated aCSF at room temperature at least 1 hr before use. Hippocampal slices posterior to the midtemporal (caudal) part of the hippocampus (Paxinos and Watson, 1986) were used in the study.

In one example, electrophysiological recordings are gathered from individual slices and subsequently transferred to a conventional submerged-type chamber and continuously superfused with, for example, oxygenated aCSF. Whole-cell recordings were generated. Patch electrodes were used, made by known methods. For whole-cell recording in voltage-clamp mode pipettes were filled with a buffered solution.

From the example outlined above, extracellular field potentials were recorded and microelectrodes known in the art were used for simultaneous recordings of multiple unit activity (e.g. MUA; 500 Hz high-pass filter), population field activity in EEG band (1-100 Hz), and fast ripple oscillations (200-600 Hz).).

In one experimental example, bumetanide was tested for suppression of a 8.5 mM potassium-induced epileptiform activity in hippocampal slices from P7-9 and PI0-12 rats. After analysis of seizure parameters in the treated hippocampus slice versus the control, the bath application of bumetanide (10 □M) strongly suppressed interictal and ictal-like epileptiform activity in P7-9 hippocampal slices (n=10; FIG. 3A, 3C) and depressed ictal-like epileptiform activity in P10-12 hippocampal slices (n=6; FIG. 3C). Averaged power of epileptiform activity was decreased by 71±2.1% (n=9, p=1.57E-12) in P7-9 rat hippocampal slices and by 41.9±1.5% (n=6, p=2.13E-11) at P10-P12 (FIG. 3C).

In another aspect of this example, pharmaceutical compositions of the present invention may be tested for effects on the GABA_{A} receptor using for example a GABA_{A}-R antagonist before, during or after treatment with the pharmaceutical composition. In one example, GABA_{A} receptor antagonists prevented the effect of bumetanide on power of epileptiform activity in the hippocampal slices from neonatal rats (FIGS. 3B and 3D). Bath application of GABA_{A}-R antagonist bicuculline (10 □M) reduced the frequency and increased the amplitude of interictal epileptiform discharges (IED) evoked by 8.5 mM [K+]0 in the hippocampal slices from P7-9 rats 9. Subsequent application of bumetanide (10 □M) in the presence of bicuculline decreased IED length by 2.2±8% (p=0.8) and increased IED frequency by 22.8+6.4% (n=6, p=0.008), but did not change averaged power of epileptiform discharges (p=0.35). Here, an inhibition of synchronous network activity by bumetanide treatment in the perinatal hippocampal slices in this in vitro experiment was observed and these affects were partially reversed after treatment with bicuculline.

In Vivo Assay Method

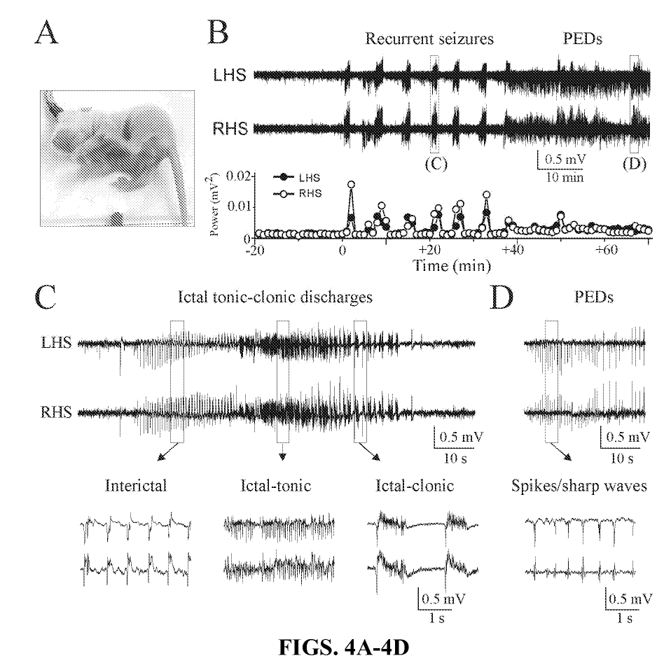

Pharmaceutical compositions of the present invention can be administered to seizure-induced rats to test the seizure modulatory properties of the pharmaceutical composition. In one exemplary method, the inhibition of NKCCl and its affect on seizures in rat pups was studied. Two days after implantation of recording electrodes, six P9-P10 rats received subcutaneous injection of KA (2 mg/kg) and six P9-10 control rats received subcutaneous 0.9% sodium chloride saline injections. 10-20 minutes after KA injection regular pattern of behavioral signs started, involving scratching-like movements of the hindpaws, loss of balance and turning on the side. Recurrent interictal and ictal electroencephalographic patterns occurred 20-80 minutes (61.2±10.4 min; mean±se) after KA injection in all rats from this experimental group (FIG. 4B). No behavioral and EEG seizures were observed in the control animals. Interictal spikes usually occurred synchronously in the left and right hemispheres. Frequency of the interictal spikes was 1.43±0.06 Hz (n=23 seizures in 6 rats). Before transition to ictal patterns, interictal spikes were usually followed by fast ripple (100-500 Hz) and gamma-frequency oscillations (20-50 Hz). Tonic-clonic seizures were the most common type of seizures induced by KA in P9-10 rats. Ictal-tonic phase lasted 12.6±1.4 s (16 seizures in 5 rats). Population spikes of 18.7±1.2 Hz were characteristic of the tonic phase of KA-induced seizures (FIG. 4C). Tonic discharges were followed by clonic bursts (FIG. 4C). Large amplitude population spikes followed by 100-200 Hz ripples and 200-1200 ms gamma frequency after discharges characterized the clonic bursts. The mean duration of the clonic phase was 36.4±5 s (25 seizures in 6 rats). The ictal patterns were followed by postictal depression (FIG. 4 B-4C). During ictal EEG activity, motor activity consisted of scratching, jerky movements, turning on the side or on the back and frequent tail shaking. Five to ten recurrent seizures with an average interval 379±24.5 s were followed by periodic epileptiform discharges (PLEDs) (FIG. 4D).

Bumetanide for Neonatal Seizure Therapy

In one exemplary method, bumetanide was tested for its seizure modulatory affects on KA-seizure induced rats. The KA-exposed rat model detailed previously is an established model for seizure studies and is believed to parallel human neonatal seizures. Any one of the pharmaceutical compositions of the present invention can be tested for inhibitory affects on seizures of the KA rat seizure induced model.

In one exemplary experiment, a pharmaceutical composition of the present invention comprising bumetanide was compared to phenobarbital for effects on KA-induced rat seizures. Two days after implantation of recording electrodes, twenty-two P9-12 age-matched rats received subcutaneous injection with KA (2 mg/kg). Ten minutes after KA injection ten rats received intra-peritoneal (i.p.) 0.9% sodium chloride saline injection (control), six rats received i.p. injection with phenobarbital (25 mg/kg) and six rats—with bumetanide (0.1-0.2 mg/kg).

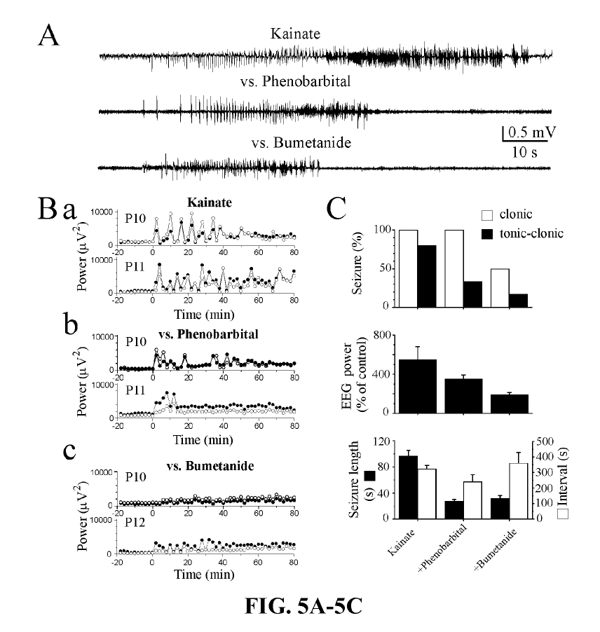

Following KA and sodium chloride saline injections, recurrent interictal and ictal-clonic EEG activity occurred in 100% of the rats in this experimental group (FIG. 5C). Frequency of interictal spikes was 1.37±0.07 s (n=33 seizures in 10 rats). The mean duration of single ictal episode including interictal and ictal phases was 96.8±8.6 s (n=38 seizures in 10 rats). The mean interval between seizures was 323±23 s. In eight of ten rats seizures included 18.2±1.1 Hz tonic discharges lasting 11.9±1.3 s (n=23 seizures). The ictal-clonic pattern lasting 40.3+5.1 s (n=38) was characterized by primary population spikes followed by secondary after discharges. The power of the EEG activity in a 20 minute window following seizure onset increased by 444.7±136.3% compared to the pre-ictal baseline period (p=3.67E-4; 19 EEG recordings in 10 rats) (FIG. 5B). These parameters reflect various episodes in rat-induced seizures that mimic episodes in neonatal seizures. Therefore, this model of KA-induced rat seizures allows for the evaluation of pharmaceutical compositions of the present invention for efficacy in the treatment of seizures, particularly neonatal seizures.

In the 6 rats in which KA treatment was followed by phenobarbital treatment, 100% of the animals developed recurrent interictal and ictal-clonic activity. The interictal phase was characterized by population spikes at 1.56+0.08 Hz, followed by gamma frequency afterdischarges before transition to ictal-clonic phase. The mean duration of single ictal episode was 27.4±2.6 s (n=19). The mean interval between seizures was 241.3±45.8 s. The ictal-tonic pattern lasting 5.2±1.4 s (n=4 seizures) occurred in two rats (33.3%; n=2 of 6). The frequency of the ictal-tonic discharges was 14±3 Hz. The ictal-clonic pattern lasting 12±1.1 s (n=19) was characterized by primary population spikes followed by secondary afterdischarges (FIG. 5C). The power of EEG activity in the 20 minute window following seizure onset increased by 248±40.8% (p=5.4E-4; 12 EEG recordings in 6 rats) (FIG. 5B).

In the 6 rats in which KA treatment was followed by bumetanide treatment, periodic epileptiform discharges occurred in all six rats. Three rats from this experimental group were observed to have EEG clonic seizures (50%/o) as opposed to the 100% seen in phenobarbital treated rats. One rat from these three also exhibited tonic-clonic seizures (16.7%) (FIG. 5C). The frequency of interictal spikes preceding transition to ictal activity was 1.42+0.07 Hz (12 seizures). The mean duration of seizure including interictal and ictal phases was 32±3.9 s (n=12 seizures in 3 rats). The ictal-tonic phase lasted 9±1.9 s (n=4 seizures in 1 rat) and ictal-clonic phase—12.5±1.1 s (n=12 seizures in 3 rats). The mean interval between recurrent seizures was 360.1±68.6 s. Power of EEG activity in the 20 minute window following seizure onset increased by 88.7±24.5% (p=0.01; 111 recordings in 6 rats) (FIG. 5B). Bumetanide treatment of KA-induced rats reduces seizure disorders in this animal model compared to rats treated by conventional therapy such as phenobarbital. The efficacy of pharmaceutical compositions of the present invention to modulate the occurrence of seizures, such as neonatal seizures, can be tested by this model.

Chloride Ion Cotransporter Expression in Human and Rat Cortex

To determine if the sodium/potassium/chloride cotransporters are differentially expressed in mature versus immature cortex samples, in one exemplary method, the level of expression of sodium/potassium/chloride cotransporters in the cortex was measured in rat and human neonatal samples and compared to mature cortex samples. In one embodiment, the expression of these transporters may be compared in control samples versus samples treated with any pharmaceutical composition of the present invention to assess any changes in level of expression of these transporters.

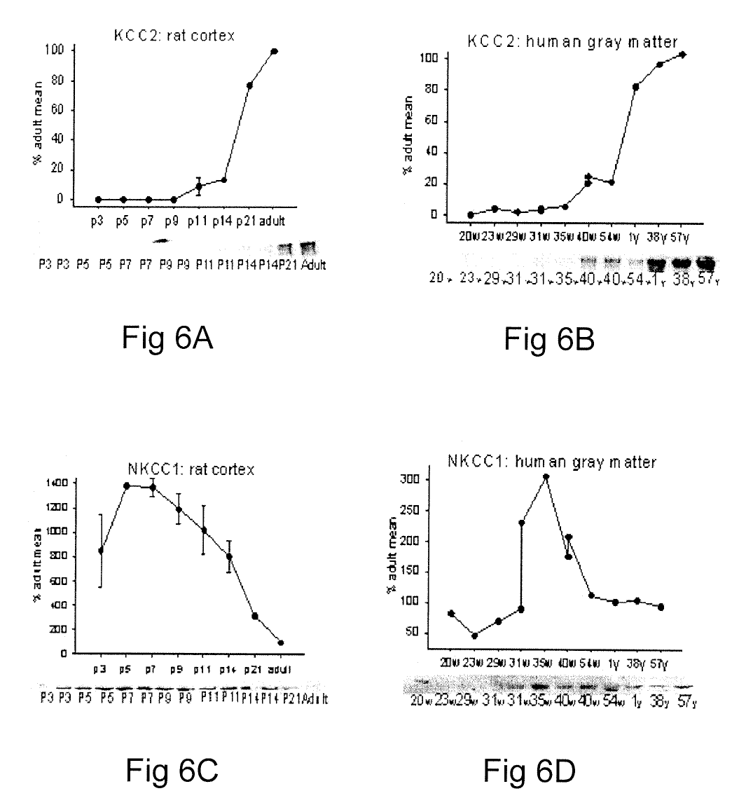

In one example, NKCCl vs. KCC2 transporter expression in human and rat cortex demonstrates that chloride transport in perinatal human cortex is as immature as in the rat. KCC2 expression in rat cortex is demonstrated (FIG. 6A-6D), as a percentage of adult levels, at different postnatal ages. Western blotting techniques were used to measure transporter expression of KCC2 and NKCCl (FIG. 6A-D, inset). KCC2 expression, as a percentage of adult expression, in human cortical brain tissue is shown in FIG. 6B. This experiment demonstrates that near term, KCC2 expression is only 5-20% of adult expression levels. NKCCl levels in rat cortex are illustrated in FIG. 6C. NKCCl levels in human tissue peak near term as illustrated in FIG. 6D. These experiments identify the differential expression of the two ion transporters and indicate at least one system that affects human neonatal seizures treatment by GABA stimulating anticonvulsants. The poor response is likely due to the delicate balance of chloride ions in neuronal cells and immature chloride ion transport out of neonatal neurons due to low levels of expression of KCC2.

Thus, in one embodiment of the invention, a pharmaceutical composition of the present invention, such as a pharmaceutical composition comprising a chloride ion uptake modulator (e.g. a diuretic compound), can be used to modulate further chloride ion transport into neurons (e.g. neonatal neurons) of a subject suffering from a sodium/potassium/chloride cotransport disorder. In one preferred embodiment, a pharmaceutical composition comprising a diuretic such as bumetanide can be used for treating a subject experiencing a sodium/potassium/chloride cotransport mediated disorder by inhibiting the chloride ion transporter BSC2. In a preferred embodiment, a pharmaceutical composition comprising a diuretic such as bumetanide can be combined with another agent such as a GABA_{A} receptor modulator, an anticonvulsant agent, an antidiuretic agent, or a combination thereof, for treating a subject experiencing a sodium/potassium/chloride cotransport mediated disorder by inhibiting the chloride ion transporter BCS2.

It is contemplated in the present invention that the activity of a sodium/potassium/chloride cotransporter such as NKCCl and/or KCC2 can be measured and compared by methods known in the art in samples illustrated in the present invention such as in vivo and in vitro samples. In one embodiment, any pharmaceutical composition of the present invention can be introduced in vivo or in vitro to assess a modulation in activity of a sodium/potassium/chloride cotransporter and the affect of this modulation on a subject or sample experiencing a sodium/potassium/chloride cotransporter mediated disorder such as a seizure.

General Methods

Recording Parameters:

Artificial cerebrospinal fluid (aCSF) will be saturated with 100% O₂ and contain a buffer. Whole-cell recordings will be performed using a filling solution containing known in the art. For neonatal and adult experiments, the chloride ion concentration of whole-cell electrode solutions will adjusted to the steady state chloride ion determined from gramicidin recordings; this is approximately 20 mM at P7 and 6 mM at 16 weeks; methylsulfonate will be used to substitute for Cl⁻. The intracellular and extracellular solutions will be buffered. Extracellular recordings can be performed using tungsten electrode array. Recordings will be accepted when the access resistance is stable at <10 MΩ for voltage clamp experiments. Recordings using known amplifiers will be digitized.

E_{Cl} can be estimated from the reversal potential of currents evoked by GABA application to the dendrites in a known media. GABA dissolved in for example aCSF can be applied to the sample. GABA_{B} antagonists (e.g. 1 μM CGP55845A) will be present in the bath to eliminate contamination from GABA_{B} receptor activation. EGAB_{A} can be estimated from the reversal potential of GABA currents evoked after block of ionotropic glutamate receptors and GABA_{B} receptors by known methods.

Data Analysis:

Current-voltage plots can be generated by fitting the GABA_{A} receptor-mediated currents to the Goldman-Hodgkin-Katz constant field equation:
I=G×Clₒ×VF²×(1−e^{(Vo-V)F/RT})/(1−e^{-VF/RT}) 1
Clₒ is the extracellular concentration of permeant anions=extracellular Cl⁻ in these experiments. G=GABA_{A} conductance; V=membrane potential; F=Faraday's constant (96,487 C/mol), R=gas constant (8.315 J/mol/° K) and T=temperature (° K). V₀ represents the membrane potential corresponding to the zero-current condition for the constant field current equation i:
Vₒ=−(RT/F)×Log_{c}(A_{EC}/Clᵢ) 2
which is the Nernst equation (Hodgkin and Katz 1949). Charge transfer by GABA_{A} receptor-mediated postsynaptic currents (PSCs) can be calculated by numerical integration of the PSC waveform.
Estimating Vₘₐₓ of Cl⁻ Transport:

Ionotropic glutamate and GABA_{B} receptors can be blocked as described above. The resting E_{Cl} can be calculated from the I-V relationship of the currents evoked at 20 second intervals between GABA applications using equations 1-2. Ec and the GABA_{A} conductance will be calculated from equation 1. The Cl⁻ transport rate can be calculated from the rate of recovery of currents evoked by exogenous dendritic GABA application. A series of 2 GABA applications can be used. The first GABA application can be at a test potential 30 mV from E_{Cl}, and the second application can be within 5 mV of E_{Cl}. To measure NKCCl kinetics, the initial GABA-evoked current can be inward (outward Cl⁻ flux will deplete Clᵢ). For KCC2 kinetics, the initial current can be outward (inward Cl⁻ flux will load cytoplasm with Cl). The timing of the second GABA application is varied from 1-10 seconds after the initial application. Clᵢ at the time of the second GABA application can be estimated from the change in Clᵢ necessary to account for the current produced by the repeat GABA application, using the GABA conductance and steady-state E_{Cl} determined from the I-V relationship. Equation 3 is a rearrangement of an alternate formulation of equation 1 (using Clᵢ and Clₒ instead of V₀), and uses the amplitude of the current evoked by the second GABA application, I, the conductance, G, and test potential V to calculate Clᵢ:
Clᵢ=Clₒ+I×(1−e^{-VF/RT})/(−GF²V/RT×e^{-VF/RT}) 3

Data for the rate of change of Clᵢ can be fit to a monoexponential curve using least-squares fit of equation 4, where t=0 is the time at which the initial evoked current decays to 0 or when the test potential is stepped to a value near E_{Cl}. Cl_{ic} is the variation in Clᵢ between t=0 and t=∞; Clᵢ, is the constant portion of Clᵢ. (If Clᵢ changes from 8 mM at t=0 to 20 mM at steady state, Clᵢᵥ=12 mM and Cl_{ic}=8 mM). The Clᵢᵥ term is positive for re-accumulation via NKCCl and negative for Cl extrusion via KCC2. Clᵢᵥ, Cl_{ic}, and tau are fit using unconstrained least squares algorithms.
Clᵢ(t)=Cl_{ic}+/−Clᵢᵥ×(1−e^{-t/τ}) 4

The slope of equation 4 at t=0 provides the initial (maximum) velocity, Vₘₐₓ, for Clᵢ re-accumulation (NKCCl) or extrusion (KCC2). Receptor desensitization can be corrected by setting the test potential for second GABA puffs such that transport decreases the driving force and current amplitude.

Estimating Inhibition of Transport by Clᵢ:

Vₘₐₓ of Cl⁻ transport can be calculated as above. The experiment can be repeated several times, with the initial GABA applications occurring at test potentials that are either 10, 20, 30, and 40 mV away from E_{Cl} (negative to E_{Cl} for NKKCl; positive to E_{Cl} for KCC2). These different test potentials can change the size of the initial GABA_{A} current and thus alter Clᵢ by different amounts; when equations 3 and 4 are used to fit the recovery of Clᵢ vs time, 4 different Clᵢ at t=0 and 4 corresponding Vₘₐₓ estimates from the slope of Clᵢ (t) at t=0 can be used. The rate of transport of Cl⁻ as a function of its cytoplasmic concentration has been characterized for NKCCl and KCC2 using the Michaelis-Menten kinetic model ii, iii, iv, v. 1/Vₘₐₓ can be plotted vs. 1/Clᵢ (FIG. 5) to determine the effect of Clᵢ on initial transport velocity, and K_{D} can be calculated using equation 5:
1/v=K_{D}/(Clᵢ×Vₘₐₓ)+1/Vₘₐₓ 5
where Clᵢ is the calculated neuronal Cl⁻ concentration, K_{D} is the neuronal Cl− concentration at which the NKCCl or KCC2 transport rate is half-maximal, and vₘₐₓ is the maximum rate of Cl⁻ transport. Data for Lineweaver-Burke plots can be fit using a least squares algorithm.
Acute In Vivo Recordings: Recordings can be Performed in Neonatal Rats. Methods Known in the Art is Used

Chronic in vivo recordings:

EEG Electrodes will be implanted in adult (e.g. 6 week) rats as above, but the EEG electrodes will be connected to an implantable radiotelemetry transmitter (DSI) rather than an external Omnetics connector. The transmitter is placed subcutaneously on the animal and a receiving antenna plate captures the telemetry signal. The EEG signal is then continuously digitized at 250 Hz. Animal behavior and seizures can be recorded. Target camera with automatic gain control can be used. Seizures are identified and quantified using algorithms that exploit the power spectrum of the normal vs. epileptic EEG signal combined with autocorrelation of the seizure signal. Because Racine stage 3 and lower seizures are frequently not apparent on video review these will be assessed separately by known methods.

Thermodynamic Regulation of NKCCl-Mediated Cl− Transport

Methods:

Slice Preparation:

Acute hippocampal slices (400 μm) were prepared from male Sprague-Dawley rats, age postnatal day (P) 3 through P6. Slices were cut in ice-cold solution containing (in mM): 87 NaCl; 2.5 KCl; 25 NaHCO₃; 0.5 CaCl₂.2H₂O; 7 MgCl₂.6H₂O; 2.25 NaH₂PO₄.H₂O; 25 glucose; 75 sucrose; bubbled with 95% O₂/5% CO₂. Slices were allowed to recover at room temperature for at least one hour in a solution containing (in mM): 124 NaCl; 2.5 KCl; 26 HEPES; 1.25 CaCl₂.2H₂O; 4.5 MgCl₂.6H₂O; 1.75 NaH₂PO₄H₂O; 17.5 glucose; 108.5 sucrose; bubbled with 100% O₂; pH=7.4.

Extracellular Recording Solution:

Slices were perfused at >2 mL/min at 32° C. with nominally bicarbonate-free artificial cerebrospinal fluid (ACSF) containing (in mM): 126 NaCl, 2.5 KCl, 26 HEPES, 2 CaCl₂.2H₂O, 2 MgCl₂.6H₂O, 1.25 NaH₂PO₄.H₂O, and 10 glucose; pH=7.3; saturated with 100% O₂.

Gramicidin Perforated Patch:

Gramcidin stock (40 mg/mL in DMSO) was prepared daily and diluted to 80 μg/mL in patch solution which contained 150 Cl⁻ and 10 HEPES, with either 141.1 K⁺ and 8.9 Na⁺, or 150 K⁺; pH=7.2 with KOH; 290 mOsM; aliquots were stored at −20° C. Gramicidin was stored in a desiccator at 4° C. DMSO was stored with 4 Å molecular sieves to minimize water content.

Whole Cell Recordings:

Intracellular solutions were comprised of potassium gluconate and (in mM) 4 Na₂ATP, 0.3 Na₃GTP, 1 QX314, 8.9 Na⁺, 1 EGTA, 10 HEPES, 10 Cl⁻, and 2 Mg²⁺; pH=7.2; 290 mOsM.

Drugs:

The GABA_{B} receptor antagonist CGP 55845A (1 μM) was included in the bath for most experiments. Bumetanide was stored as a 50 mM stock solution in ethanol at 4° C. and then diluted to 10 μM in ACSF. Dihydroouabain (10 μM) was diluted into ACSF and bath applied.

Experimental Procedure:

Recordings were used if the access resistance was <25 MΩ for whole cell and <40 for gramicidin experiments. Recordings using a Multiclamp 700A amplifier were digitized at 10 KHz using pClamp 8.2 software (Axon Instruments). CA1 pyramidal neurons were visualized at 40× magnification using a Zeiss Axioskop with differential interference contrast (DIC) optics. Capacitance was compensated throughout the experiment.

E_{Cl} was estimated from the reversal potential of currents evoked by pressure application of 100 μM GABA to the dendrites of voltage clamped CA1 pyramidal cells (10 ms, 5 psi; Picospritzer II, Parker). Charge transfer by GABA_{A} receptor mediated currents was calculated by integration of the current waveform. GABA responses were recorded only after E_{Cl} reached a steady state, which was usually 10-15 minutes after whole cell break-in, 25-60 minutes after sealing onto the cell for gramicidin perforated patch, and 30 minutes after bumetanide application.

Action potentials in current-clamped neurons was invoked by injecting depolarizing current pulses (1.5 ms, 2 nA) at 20 Hz for 2.5 min.

Discussion

GABA inhibits neurons in the mature central nervous system by gating the influx of Cl⁻ ions. Without being bound by any theory, it is believed that GABA-mediated synaptic signaling undergoes an unusual form of activity-dependent long-term plasticity whereby the GABA currents become depolarizing due to a shift in the Cl⁻ reversal potential (E_{Cl}). This plasticity is believed to require an active Cl⁻ uptake mechanism. This example examines Cl⁻ transport by NKCC1, the principal means of inward Cl⁻ transport in neurons.

Gramicidin perforated patch recordings in young hippocampal pyramidal indicated NKCCl transport was at thermodynamic equilibrium at the steady state intracellular Cl⁻ concentration (Clᵢ). These findings imply that E_{Cl} should be sensitive to E_{Na} and E_{K}, and it was found that Na⁺-K⁺-ATPase inhibitors blocked the shift in E_{Cl} induced by action potential trains. Thus long-term changes in GABA-mediated synaptic signaling are induced by activity-dependent increases in Na⁺-K⁺-ATPase activity that are sufficient to alter E_{Cl} by changing the thermodynamic equilibrium for NKCCl.

GABA, the main inhibitory neurotransmitter in the adult brain, normally hyperpolarizes neurons by gating a net influx of anions. However, GABA depolarizes and excites neurons during development, after trauma, in human and experimental epilepsy, in models of neuropathic pain, in normal adult primary sensory neurons, and as a long-term consequence of certain patterns of neuronal activity. In these situations, it is believed that neurons accumulate intracellular Cl⁻ (Clᵢ) beyond electrochemical equilibrium so that E_{Cl} is positive to resting membrane potential, and GABA_{A} receptor activation gates a depolarizing efflux of anions.

NKCCl is a Na⁺—K⁺—Cl⁻ cotransporter expressed in neurons during early development that is thought to mediate the inward Cl⁻ cotransport responsible for Clᵢ accumulation and therefore excitatory GABA responses in neonatal neurons. It is believed that changes in NKCCl cotransport could explain activity-dependent long-term increases in E_{Cl}. In this example, NKCCl-mediated Cl⁻ cotransport and activity-dependent, persistent alterations in Cl⁻ transport in neonatal CA1 pyramidal neurons were quantified.

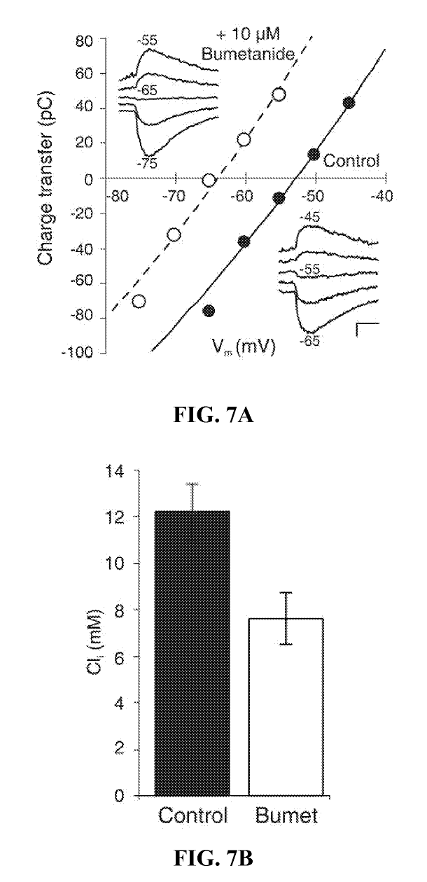

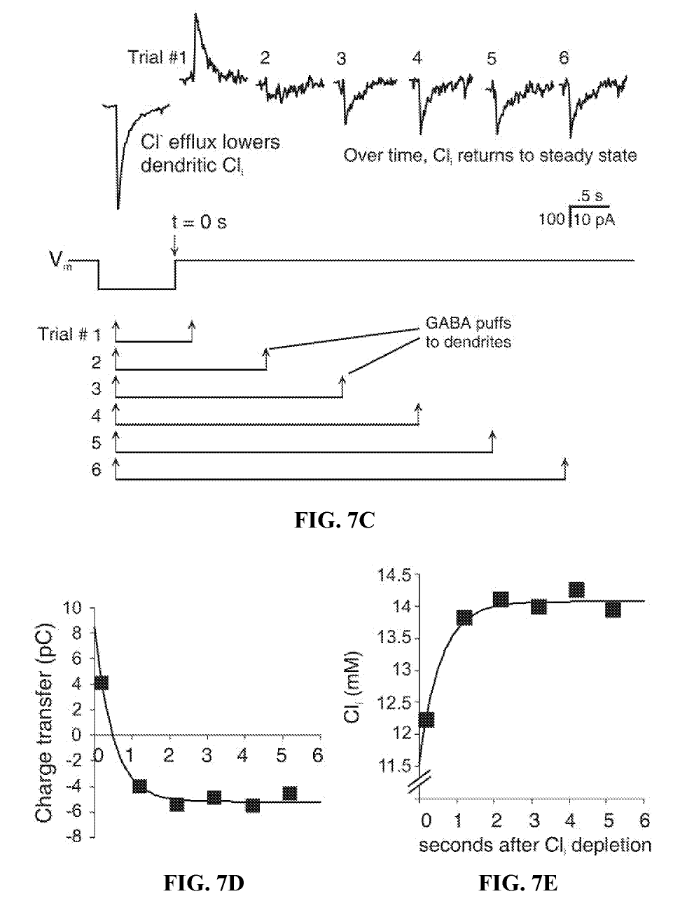

To test whether NKCCl accumulates Clᵢ above equilibrium in postnatal day 4-6 rat pups, the resting membrane potential (RMP) and E_{Cl} of hippocampal CA1 pyramidal cells were measured using gramicidin perforated patch recordings in the presence and absence of 10 μM bumetanide, a selective inhibitor of NKCCl. In nominally bicarbonate-free media, pressure application of 100 μM GABA to the dendrites ˜100 μm from the soma evoked Cl⁻ currents that reversed at −67.34±3.78 mV, corresponding to a Clᵢ of 12.00±1.47 mM (FIGS. 7A & 7B). RMP was −69.32±1.8 mV. In bumetanide, E_{Cl} was −80.14±4.61 mV, corresponding to a Clᵢ that was lowered by 37±0.05% to 7.62±1.14 mM with no change in holding current (n=9; P=0.0006). These results indicate that NKCCl is necessary for CA1 pyramidal cells to maintain E_{Cl}>RMP and thus enable depolarizing responses to GABA_{A} receptor activation.

FIGS. 7A-7D shows NKCCl activity is required to maintain elevated Clᵢ. In the presence or absence of 10 μM bumetanide, a selective inhibitor of NKCCl, GABA_{A} receptor mediated Cl⁻ conductances were evoked by pressure application of 100 μM GABA to the dendrites of a gramicidin perforated patch clamped CA1 pyramidal neuron in an acute hippocampal slice from a P5 rat. HEPES-buffered nominally CO₂/bicarbonate-free ACSF was used in all experiments to minimize the HCO3⁻ flux through the GABA_{A} channel. FIG. 7A shows selective NKCCl inhibition (open circles) hyperpolarizes E_{Cl} compared to control (filled circles). Scale bar=50 pA; 200 ms. FIG. 7B is a bar graph showing steady state Clᵢ in the presence or absence of NKCCl activity (mean±s.e.m; n=9; P=0.0006 by paired two tail t-test). FIGS. 7C to 7E are graphs showing quantification of inward Cl⁻ transport after dendritic Cl⁻ efflux. In FIG. 7C, to lower dendritic Clᵢ, Vₘ was stepped to −123 mV for one second and GABA was applied to the dendrites to elicit a large outward Cl⁻ flux. Vₘ was then stepped to −63 mV (3 mV negative to steady-state Eci) and a second GABA puff evoked a “test” current. Six trials of paired GABA puffs were performed, each with a different delay between Clᵢ depletion and the test current. FIG. 7D shows that as the interval between the Clᵢ depletion and test current increases, the charge transfer of the test currents returns to steady state. In FIG. 7E, Clᵢ is calculated from the shift in E_{Cl} that accounts for the direction and charge transfer of each test current. Fit lines are single exponentials.

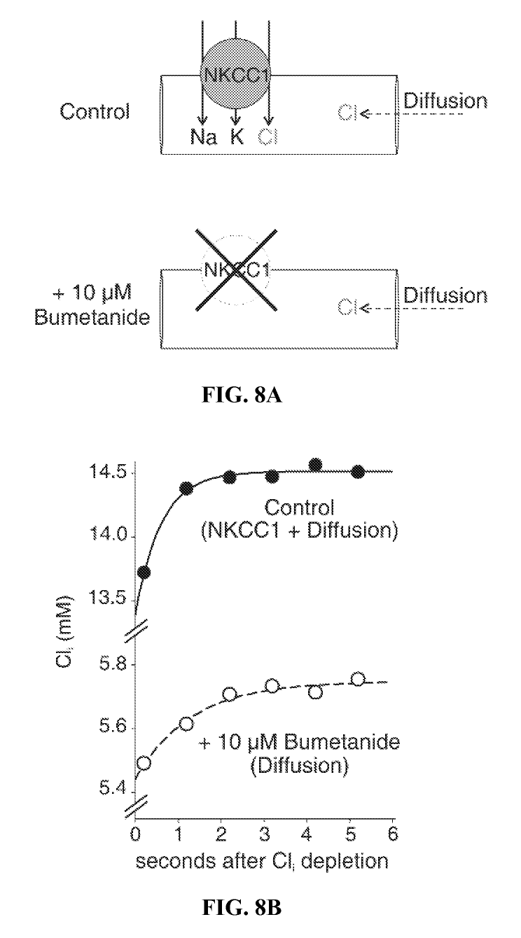

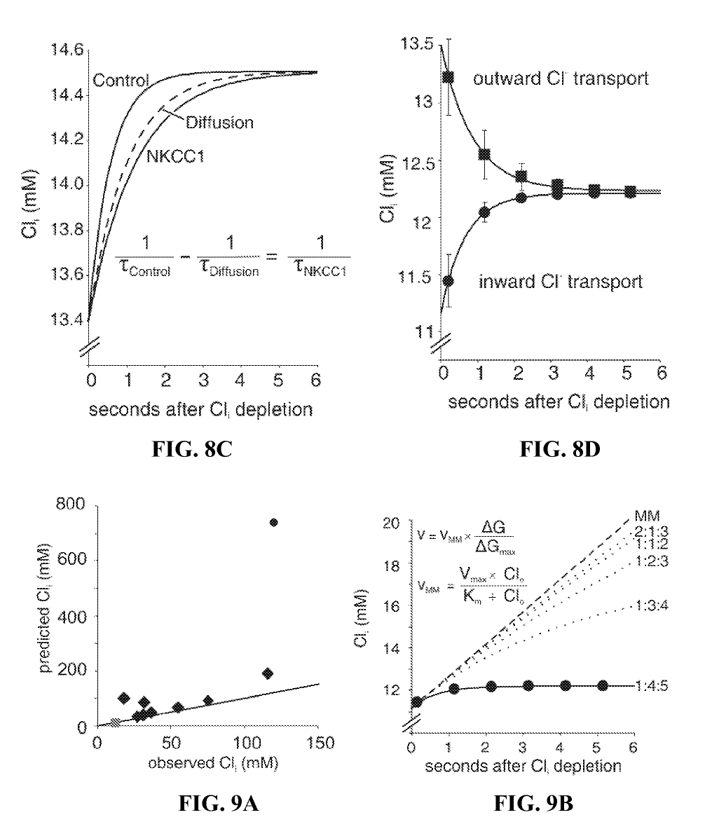

To quantify NKCCl-mediated Cl⁻ transport into neurons, the rate at which E_{Cl} returned to baseline after an outward Cl⁻ transient was measured. For the cell in FIG. 7C, Vₘ was stepped to −123 mV and GABA was puffed onto the dendrites to induce Cl⁻ efflux that was large enough to deplete Clᵢ and thereby alter E_{Cl}. Vₘ was then stepped to −63 mV (3 mV negative to steady state E_{Cl}) and a “test” current was evoked with a second GABA application at varying time intervals following the first Clᵢ-depleting GABA current. Clᵢ was calculated at each time interval based on the shift in E_{Cl} implied by the change in the sign and amplitude of each test current (FIGS. 7D & 7E). To determine the NKCCl-specific component of Clᵢ recovery, the experiment was repeated in the same cell after blocking NKCCl with 10 μM bumetanide. FIG. 8B shows the recovery of Clᵢ after depletion for control (filled circles) and with NKCCl activity blocked (open circles), each fit with a single exponential. The Clᵢ recovery that takes place when NKCCl was blocked was also a monoexponential process that was well described by dendritic Cl⁻ diffusion. First order rate constants (k=τ⁻¹) were additive, so 1/τ_{NKCCl} was calculated by subtracting 1/τ_{diffusion} from 1/τ_{control} (FIG. 8C). Following dendritic ΔClᵢ of −1.04±0.27 mM, NKCCl transported Cl⁻ into the cell with r=0.58±0.08 s and Vₘₐₓ=1.58±0.28 mM/s (FIG. 8D, circles, n=5). When Clᵢ was transiently increased by evoking the first GABA-gated current at a test potential positive to E_{Cl}, first-order NKCCl-mediated transport was again demonstrated in the opposite direction (ΔClᵢ=+1.31±0.37 mM; τ_{NKCCl}=0.85±0.33 s; Vₘₐₓ=1.60±0.51 mM/s; FIG. 8D, squares, n=3). For both inward and outward Cl⁻ transport, NKCCl activity caused Clᵢ to relax to the same steady-state value of 12.22±1.22 mM.

Discussion of FIGS. 8A-8D

FIGS. 8A-8D show quantification graphs of NKCCl-mediated Cl⁻ transport. FIG. 8A is a graph showing that after an outward Cl⁻ transient, Clᵢ returned to steady state via NKCCl transport and dendritic diffusion. When NKCCl was blocked with 10 μM bumetanide, the increase in Clᵢ back to steady state was well-described by Cl⁻ diffusion alone. FIG. 8B is a graph showing Clᵢ as a function of time after an outward Cl⁻ transient for control (filled circles) and with NKCCl blocked (open circles) for a single neuron; each was fit to a single exponential. In FIG. 8C, the r of NKCCl Cl⁻ transport was determined by subtracting first order rate constants (k=1/r). FIG. 8D is a graph showing that following dendritic Cl⁻ efflux, NKCCl returned Clᵢ to steady state by inward Cl⁻ transport (n=5). Following dendritic Cl⁻ influx, NKCCl returned Clᵢ to steady state via outward Cl⁻ transport (n=3). Both inward and outward Cl⁻ transport recovered to the same steady state Clᵢ (P=0.82 by unpaired two tail t-test). Inward and outward transport data were corrected for diffusion as in FIGS. 8B and 8C and are presented as mean±s.e.m.

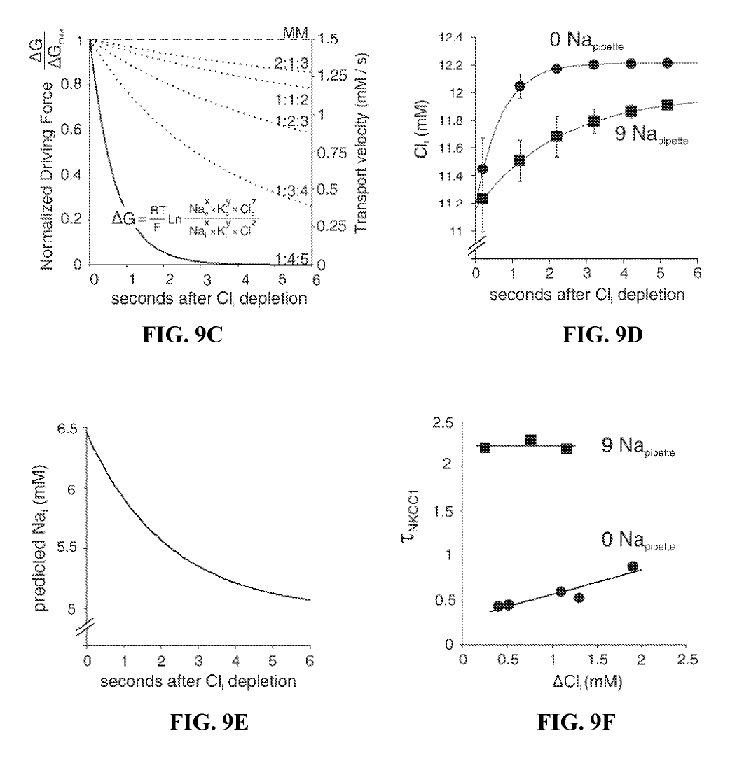

Discussion of FIGS. 9A-9F

NKCCl Cl⁻ transport was thermodynamically regulated. FIG. 9A is a graph showing that predicted Clᵢ for NKCCl at thermodynamic equilibrium correlated with previously reported transport stoichiometries and Clᵢ. Na:K:Cl ratios: diamonds, 1:1:2; circle, 2:1:3 (squid giant axon); blue square, 1:4:5 (data from FIGS. 7A-7D). The line represents unity. FIG. 9B is a graph showing NKCCl mediated inward Cl⁻ transport (same data as in FIG. 8D: filled circles, mean±s.e.m.), with the transport velocity (v) calculated according to Michaelis-Menten kinetics (MM, dashed line), or as the product of a Michaelis-Menten conductance term (ν_{MM}) and a driving force term (ΔG/ΔGₜ₌₀) for various Na⁺:K⁺:Cl⁻ transport stoichiometries (ratios 2:1:3, 1:1:2, 1:2:3, and 1:3:4, dotted lines; 1:4:5, solid line). FIG. 9C is a graph showing normalized driving force (left ordinate) and transport velocity (right ordinate) as a function of time for each Cl⁻ transport stoichiometry. FIG. 9D is a graphs showing NKCCl mediated Cl⁻ transport with pipette [Na⁺] (Napipette)=0 mM (filled circles, n=5) or 9 mM (filled squares, n=3); data are mean±s.e.m, fit lines calculated as in 3a with the 1 Na⁺:4 K⁺:5 Cl⁻ transport stoichiometry. FIG. 9E is a graph showing that the slow initial velocity and r of NKCCl Cl⁻ transport with 9 mM Naₚᵢₚₑₜₜₑ can be accounted for by a transient decrease in the free energy available for transport (FIG. 9B) that arises from a Naᵢ transient that resolves with a time constant of 1.85 s. FIG. 9F is a graph showing that with 9 mM Naₚᵢₚₑₜₜₑ (squares, R²=1×10⁻⁶) NKCCl Cl⁻ transport rate was independent of ΔClᵢ and with 0 mM Naₚᵢₚₑₜₜₑ (circles, R²=0.85), larger Clᵢ depletions correlated with slower NKCCl Cl⁻ transport. Each data point represents one cell; fit lines are linear regressions.

The mechanism by which NKCCl-mediated Cl⁻ transport sets Clᵢ is currently unknown. In some preparations, elevated Clᵢ inhibits further NKCCl Cl⁻ transport, but this effect occurs at Clᵢ that are an order of magnitude higher than observed in developing neurons (FIG. 9A), and NKCCl inhibition by Clᵢ was not consistent with the observed rapid outward transport following transient increases in Clᵢ (FIG. 8D). Michaelis-Menten models are often used to model Cl⁻ transport, but do not explain the observed steady state Clᵢ, even when modified to include non- or uncompetitive antagonism by Clᵢ (FIG. 9B). One possibility is that NKCCl is at thermodynamic equilibrium at the steady-state Clᵢ. The symmetric monoexponential relaxation of Clᵢ back to the steady state value after perturbation from either direction (FIG. 8D) suggested a return to thermodynamic equilibrium. As shown in FIG. 9A, in many preparations NKCCl was at or near thermodynamic equilibrium at the measured Clᵢ. Developing neurons have a lower Clᵢ than most other cell types. Thus thermodynamic equilibrium would require a different transport stoichiometry than what has been reported to date for NKCCl, which includes Na:K:Cl ratios of 1:1:2 and 2:1:3. An NKCCl transport stoichiometry of 1 Na⁺:4 K⁺:5 Cl⁻ in developing pyramidal cells predicts thermodynamic equilibrium at a physiologically reasonable Naᵢ of 4-5 mM and a Clᵢ equal to the experimentally observed steady state (FIG. 9B). If NKCCl-mediated Cl⁻ transport in P4-6 hippocampal pyramidal cells is modeled as the product of a Michaelis-Menten conductance term and a driving force term based on a 1:4:5 stoichiometry, then the reduced transport rates as Clᵢ approaches steady state are well-described by the reduction in free energy available to drive transport as NKCCl-mediated Cl⁻ transport approaches thermodynamic equilibrium (FIG. 9C).

It is believed that if NKCCl transport was thermodynamically limited, changing the transmembrane sodium or potassium gradients would alter the available free energy and thereby alter Cl⁻ transport. In this example, the perforated patch Clᵢ depletion/recovery experiments were repeated using a pipette solution containing Na⁺ (9 mM). The steady state Clᵢ was not significantly increased (9 mM Napipette: Clᵢ=13.94±1.11 mM (n=6); 0 mM Naₚᵢₚₑₜₜₑ: Clᵢ=13.03±1.48 mM (n=8; P=0.65). However, RMP was significantly more negative with 9 mM Naₚᵢₚₑₜₜₑ (−73.98±1.25 mV, n=6) than with 0 mM Naₚᵢₚₑₜₜₑ (−66.1±1.5 mV, n=8; P=0.002) and NKCCl-mediated Cl⁻ transport was significantly slower (τ_{NKCCl}=2.24±0.03 s, n=3) vs. τ_{NKCCl} with 0 mM Naₚᵢₚₑₜₜₑ (τ_{NKCCl}=0.58±0.08 s, n=5; P=0.000006; FIG. 9D). Dialysis from the pipette solution was progressively less effective at changing ionic equilibria in more distal dendrites due to transmembrane transport along the length of the dendrite. Na⁺ export along the length of the dendrite, which was believed to be via an electrogenic transporter such as Na⁺,K⁺-ATPase based on the increased membrane potential measured with 9 mM electrode solutions, could leave Naᵢ and the free energy available for NKCCl unchanged at the dendritic location where no change in E_{Cl} was measured. In such case, the combined Naᵢ loads from the pipette and NKCCl-mediated Na⁺ influx was expected to lead to Naᵢ transients during periods of high NKCCl-mediated transport. The lower initial velocity and transport rate of NKCCl when 9 mM Na⁺ was included in the perforated patch pipette were consistent with the predicted dendritic Naᵢ transients (FIG. 9E). Further, reduced transport rate was correlated with the size of the initial Clᵢ transient in 0 mM Na⁺ pipette solutions, which also suggested that Naᵢ transients limited NKCCl transport velocity in a manner consistent with thermodynamically-limited NKCCl transport (FIG. 9F). No significant relation between Clᵢ transient size and the reduced NKCCl transport rate was observed with 9 mM pipette Na⁺, consistent with Na⁺ transport that was already saturated by the combination of the pipette Na⁺ load and the NKCCl-mediated Na⁺ influx triggered by the smallest observed Clᵢ transients.

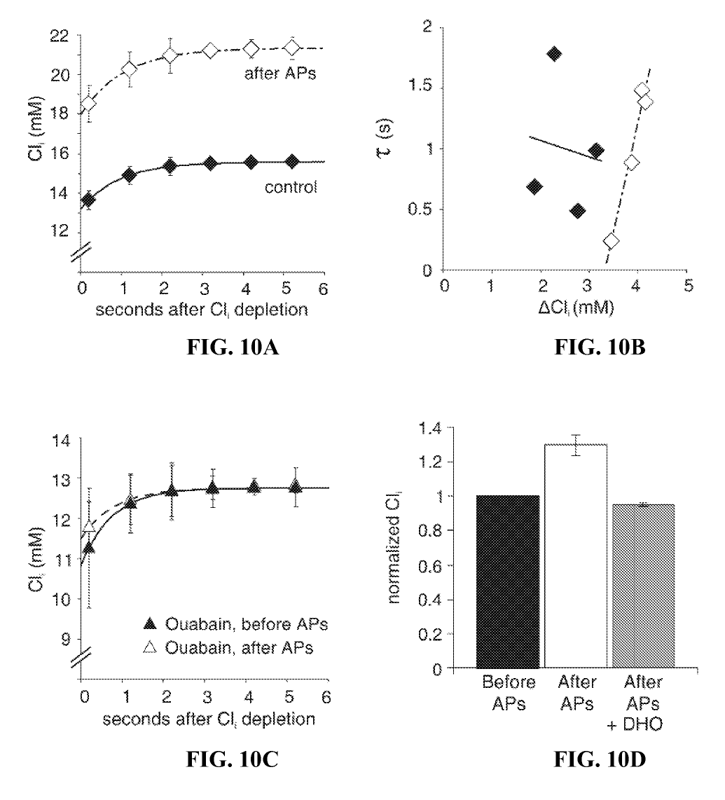

Persistent increases in Clᵢ and the GABA reversal potential have been observed following trains of action potentials. Action potential trains have long been known to cause long-lasting increases in the activity of Na⁺—K⁺-ATPase and corresponding changes in RMP. If NKCCl-mediated transport were thermodynamically limited, then the changes in E_{Cl} observed following action potential trains could be due to altered Na⁺ and K⁺ transmembrane gradients. Without being bound by any theory, it is believed that of the two cations, Naᵢ would be most labile and would have the most influence on the Clᵢ at which NKCCl-mediated transport reached equilibrium. Steady-state E_{Cl} and Cl⁻ transport kinetics were measured before and after a 20 Hz, 2.5 minute train of action potentials. The postsynaptic spiking caused Clᵢ to increase by 3.74±0.54 mM (FIG. 10A, n=3, p=0.02). As in previous experiments with 9 Na⁺ in the pipette, there was no noticeable correlation between the τ describing the Cl⁻ transport rate and ΔClᵢ in control conditions. However, after the train of action potentials, r was positively correlated with ΔClᵢ (FIG. 10B), consistent with an increased rate of Na⁺ clearance such that the Naₚᵢₚₑₜₜₑ and Na⁺ imported by NKCCl was no longer altering the transmembrane Na⁺ gradient in a manner that limited the rate of Cl⁻ transport following smaller Cl⁻ transients.

It is believed that if an activity-dependent increase in Na⁺-K⁺-ATPase activity was responsible for altering the Naᵢ and thus the NKCCl reversal potential, then blocking Na⁺-K⁺-ATPase would prevent changes in E_{Cl} due to trains of action potentials. The postsynaptic action potential trains were repeated in the presence of the selective Na⁺-K⁺-ATPase inhibitor dihydroouabain (DHO; 10 μM). In the presence of DHO, action potential trains had no effect on Clᵢ (P=0.27) nor did the trains affect Cl⁻ transport rates (FIG. 10C; n=4; P=XX). These results indicate that an activity-dependent increase in Na⁺-K⁺-ATPase activity alters the transmembrane gradients of monovalent cations such that a new equilibrium point was reached for NKCCl-mediated inward Cl⁻ transport.

Discussion of FIGS. 10A-10D

FIGS. 10A-10D show activity-dependent increases in Clᵢ. FIG. 10A is a graph showing that following a train of action potentials in the recorded cell (20 Hz, 2.5 min), Clᵢ reached a new steady state. FIG. 10B is a graph showing Cl⁻ transport rate as a function of the magnitude of Cl⁻ depletion before (filled diamonds, R²=0.02) and after postsynaptic action potentials (open diamonds, R²=0.97) for the cell in FIG. 10A. (n=3 cells, P=0.02) FIG. 10C is a graph showing that repetitive postsynaptic spiking had no effect on Cl⁻ transport when the experiment was repeated in the presence of 10 μM dihydroouabain (DHO), a selective Na⁺-K⁺-ATPase inhibitor. FIG. 10D is a graph in which Clᵢ was normalized to value before action potentials. Data shown as mean±s.e.m. In FIGS. 10A and 10C, data are presented as mean±s.d.

From these data it can be concluded that in immature hippocampal CA1 pyramidal cells, NKCCl-mediated transport was thermodynamically limited. The finding that NKCCl was at thermodynamic equilibrium at the steady state Clᵢ suggests caution in the interpretation of gramicidin perforated patch recordings, because these recordings may indirectly alter E_{Cl} by altering Na⁺ and K⁺ gradients. The results further indicate that long-term plasticity of GABA signalling due to changes in E_{GABA} can be effected by activity-dependent changes in Na⁺-K⁺-ATPase activity, which alter the transmembrane equilibrium of NKCCl-mediated Cl⁻ transport. These results suggest not only long term changes in E_{GABA} after trains of action potentials, but also suggest BDNF-dependent changes in E_{GABA} as well, in light of observation of BDNF-mediated increases in Na⁺-K⁺-ATPase activity. The results are also consistent with circadian fluctuations in E_{Cl} and NaKATPase activity observed in hypothalamic neurons. This example shows direct involvement of Na⁺-K⁺-ATPase in long-term synaptic plasticity.

Alteration of Cl⁻-Transport Enhances Efficacy of Barbiturates in Early-Life Seizure Therapy

High expression level of the Na⁺ K⁺-2Cl⁻ (NKCCl) co-transporter in immature neurons causes the accumulation of chloride ions and a depolarized Cl⁻ equilibrium potential (E_{Cl}). This is linked to a higher seizure propensity and poor EEG response in neonates to the barbiturates and benzodiazepines, conventional anticonvulsant drugs that target Cl⁻-permeable GABA_{A}-receptor (GABA_{A}-R) operated channels. This example shows that pharmacological blocking of the NKCCl by bumetanide can enhance the anticonvulsant action of phenobarbital by alteration of Cl⁻-transport. Recurrent ictal-like seizures were induced by low-Mg⁺ ACSF in intact hippocampal preparations in vitro. In the majority of experiments (70%) phenobarbital significantly reduced seizure frequency but had no significant effect on seizure power. Bumetanide decreased both seizure frequency and seizure power. Bumetanide in combination with phenobarbital much strongly reduced seizure frequency as well as completely abolished seizures in 75% hippocampi. Thus, alteration of Cl⁻-transport by bumetanide enhances anticonvulsant action of phenobarbital indicating that bumetanide in combination with GABA-enhancing anticonvulsants can be used in combination to improve the therapy of early-life seizures.

Results

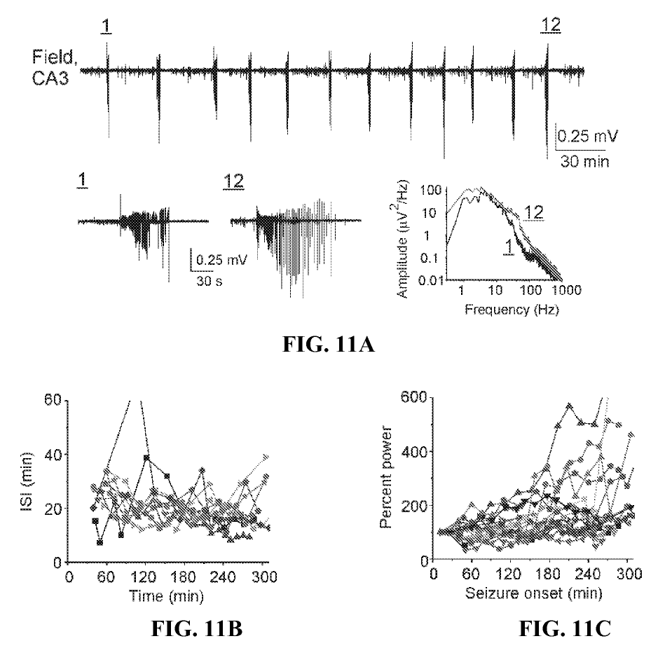

FIGS. 11A-11C are graphs of low-Mg²⁺πinduced recurrent seizures in the intact hippocampus in vitro. FIG. 1I A is a graph showing that continuous superfusion of low-Mg²⁺ ACSF resulted in recurrent tonic-clonic epileptiform activity. FIG. 11A also shows extracellular field potential recording in the CA3 pyramidal cell layer from the temporal pole of intact hippocampus preparation from a P5 rat. First and last seizures from 5 hour recording are shown on an expanded time scale. Right, corresponding power spectra in the 0.1-1000 Hz frequency band. FIG. 11B is a graph showing that inter-seizure intervals (ISI) gradually decreased following continuous application of low-Mg²⁺ ACSF. Summary data are taken from ten hippocampi. FIG. 11C is a graph showing that power of recurrent seizures gradually increased following continuous application of low-Mg²⁺ ACSF. Power of the first seizure in each recording was considered as 100%. Data are taken from 18 recordings in 10 preparations from P4-6 rats.

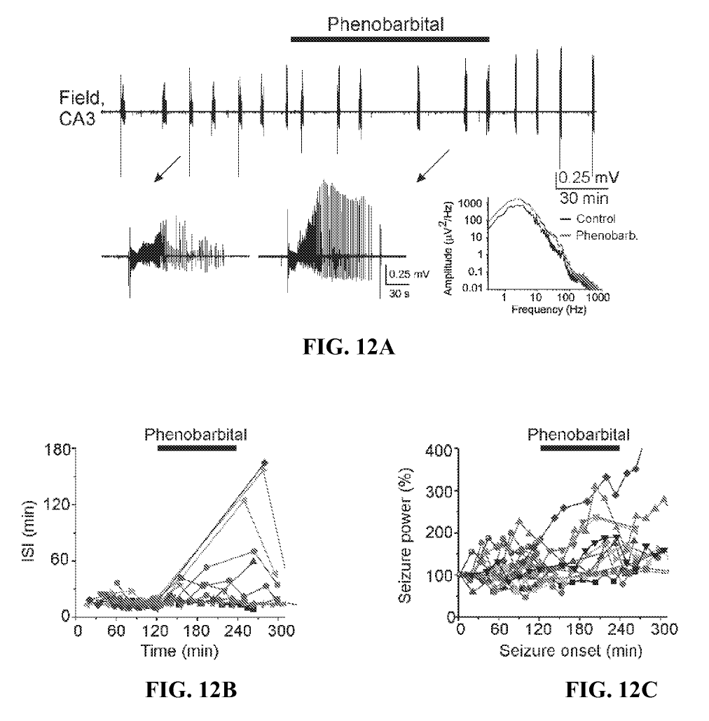

FIGS. 12A-12C are graphs showing low efficiency of phenobarbital in neonatal seizures. FIG. 12A is a graph showing extracellular field potential recording in the CA3 pyramidal cell layer in the intact hippocampus of a P5 rat. Continuous application of low-Mg²⁺ ACSF induced recurrent tonic-clonic seizures. Phenobarbital (100 μM) was applied for 120 min period that significantly covers the mean interval between seizures in control. FIG. 12A also shows graphs of two of the ictal-like events before and during application of phenobarbital shown on an expanded time scale. The right portion corresponds to power spectra in the 0.1-1000 Hz frequency range. FIG. 12B shows a graph of inter-seizure intervals before, during and after phenobarbital application. Phenobarbital increased the ISIs or abolished the seizures (ISI>120 min) suggesting its anticonvulsant effect. Data are from ten experiments that were combined. FIG. 12C is a graph showing power of recurrent seizures before, during and after phenobarbital application. Power of the first seizure in each recording was considered as 100%. Seizures were not depressed by phenobarbital indicating its low efficiency.

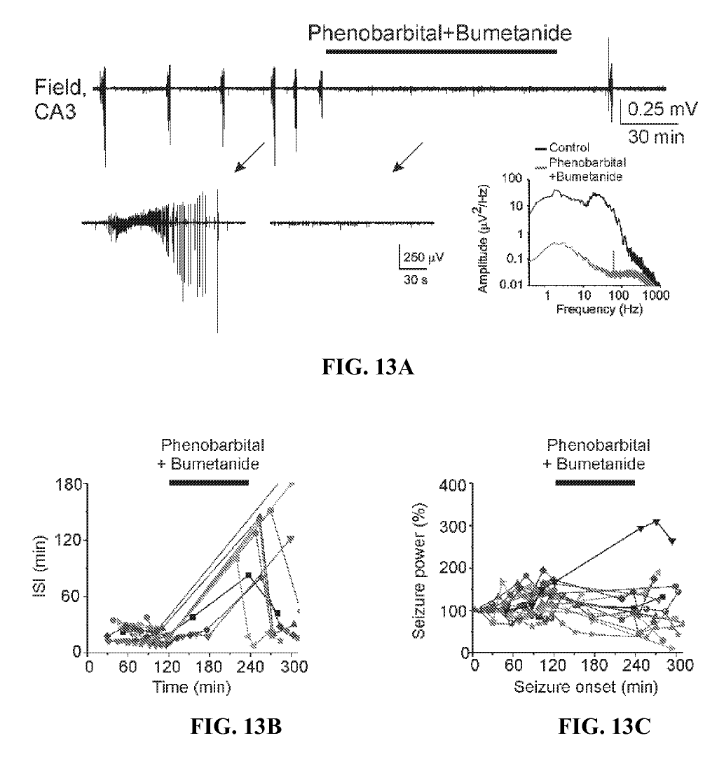

FIGS. 13A-13C are graphs showing that alteration of Cl⁻-transport by bumetanide enhanced efficacy of phenobarbital in neonatal seizures. FIG. 13A is a graph of extracellular field potential recording in the CA3 pyramidal cell layer in the intact hippocampus of a P5 rat. Application of low-Mg²⁺ ACSF induced recurrent seizures. Continuous 120 min application of phenobarbital (100 μM) in combination with bumetanide (10 μM) suppressed seizures. FIG. 13A also shows the ictal-like activity and extracellular field potential activity before and during application of drugs. These are shown on an expanded time scale. The right portion of FIG. 13A is a power spectra of extracellular field potential activity before and during application of drugs. FIG. 13B is a graph showing inter-seizure intervals before, during and after application of drugs. In 75% of experiments seizures were abolished (ISI>120 min) by simultaneous application of phenobarbital and bumetanide. FIG. 13C is a graph showing power of recurrent seizures before, during and after simultaneous application of phenobarbital and bumetanide. Power of the first seizure in each recording was considered as 100%. Seizures were depressed by combination of drugs.

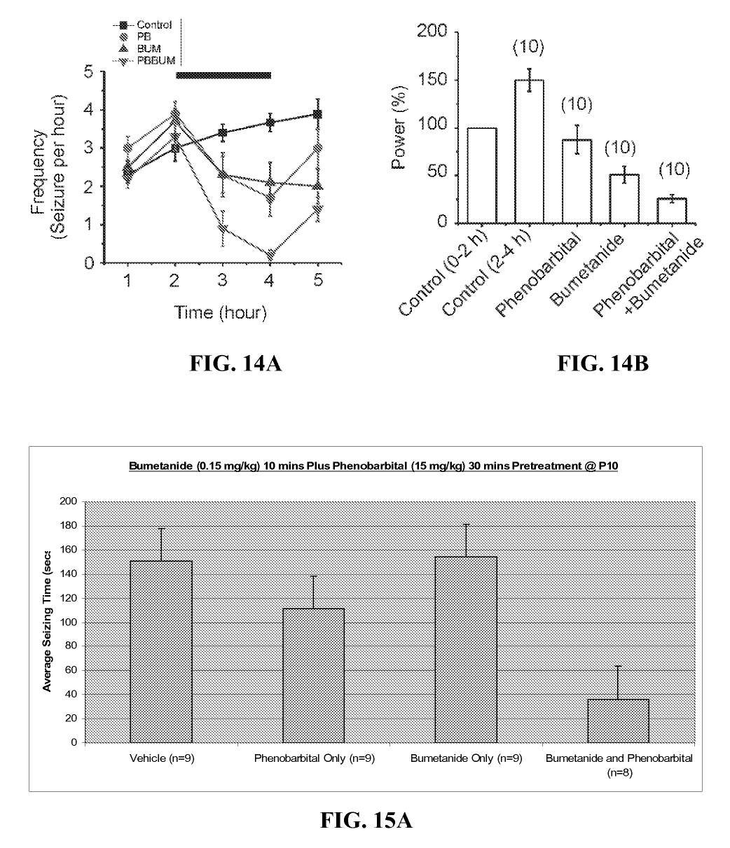

FIGS. 14A and 14B are graphs showing alteration of Cl⁻-transport for early-life seizure therapy. FIG. 14A shows a graph for mean frequency of recurrent seizures in control low-Mg²⁺ ACSF recordings (n=10) and before, during and after drug applications (n=10 for each drug or combination of drugs tested). Black bar indicate 2 hour period of the drug applications (PB—phenobarbital (100 μM); BUM—bumetanide (10 μM); PBBUM—phenobarbital (100 μM)+bumetanide (10 μM)). FIG. 14B shows a graph of mean power of extracellular field potential activity over 2-hour windows in control low-Mg²⁺ ACSF recordings and before and during application of the drugs. Data represents combination of 10 experiments. Power over 0-2 hour windows was considered 100%.

Bumetanide Study Methods

This example provides the in vivo data showing the difference in seizures between neonatal rats treated with Phenobarbital vs. Phenobarbital and Bumetanide

Animals

Litters of male Long-Evans hooded rats from Charles River Laboratories (Wilmington, Mass., USA) were used in this study. Each litter was divided into five treatment groups: no hypoxia control, hypoxia/DMSO in PBS, hypoxia/Bumetanide only, hypoxia/Phenobarbital only, and hypoxia/combination of Bumetanide and Phenobarbital. One to two litters were used for each run of the experiment, and each litter was housed together until the weaning age of P21.

Seizure Induction: Hypoxia Model

Rats at P10 (˜18 to 22 grams) were exposed to global hypoxia for 15 minutes in an airtight chamber into which N₂ gas was rapidly infused. The oxygen concentration was maintained at 7% for 8 minutes, 5% for 6 minutes, and 4% for 1 minute before termination of hypoxia, during which the animals were immediately removed from the chamber and exposed to room air. For each animal during hypoxia, a record of the total number of myoclonic seizures and the total number and length of time in seconds of tonic clonic seizures were kept. For analysis purposes, time lengths of each tonic clonic seizure for each animal were pooled to obtain total seizure time. A myoclonic seizure was identified as individual sudden jerks/jumps; tonic clonic seizures were characterized by involuntary swaying/shaking of the head and limbs. Littermate controls not undergoing hypoxia were kept at room air. The body temperature of all animals was maintained at 34-40° C. on a warming blanket; the temperatures of each animal were also recorded before and after submission to hypoxia. Rat pups from each litter were all removed from and returned to the mother rat at the same time; each litter remained together.

Drug Administration

Bumetanide was dissolved in 1×PBS at a concentration of 0.03 mg/mL. Phenobarbital was dissolved in ddH₂O at 3 mg/mL. Vehicle solution consisted of DMSO dissolved in 1×PBS at 0.03 mg/mL. All treatments were injected intraperitoneally (ip) at a dose volume of 0.1 ml per 20 grams of rat weight. For vehicle groups undergoing hypoxia, a dose of 1×PBS was injected 30 minutes prior to hypoxia, followed by a dose of vehicle solution 10 minutes prior to hypoxia. Phenobarbital-only groups received Phenobarbital 30 min prior and vehicle solution 10 min prior. Bumetanide-only groups received 1×PBS 30 min prior and Bumetanide 10 min prior. Combination-treatment groups received Phenobarbital 30 min prior and Bumetanide 10 min prior.

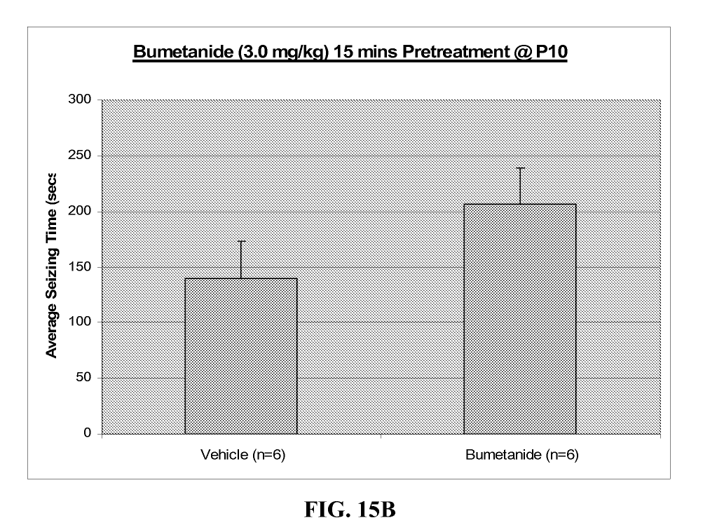

Results of this study is graphically illustrated in FIGS. 15A and 15B. FIG. 15A is a graph showing average seizing time (in secs) in rats treated with placebo (vehicle), Phenobarbital only (15 mg/kg), Bumetanide only (0.15 mg/kg) and a combination of Bumetanide (0.15 mg/kg) and Phenobarbital (15 mg/kg). As shown in FIG. 15A, combination of Bumetanide and Phenobarbital significantly reduced the average seizing time. In contrast, as shown in FIG. 15B, Bumetanide alone did not significantly reduce the average seizing time.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references disclosed herein are incorporated by reference in their entirety.

What is claimed is:

1. A method for treating a sodium potassium chloride cotransport mediated disorder comprising administering to a subject in need of such a treatment, a therapeutically effective amount of a diuretic compound.

2. The method of claim 1, wherein the sodium potassium chloride cotransport mediated disorder is seizure, epilepsy, trauma, or a disease associated with a hypoxic-ischemic event.

3. The method of claim 2, wherein the sodium potassium chloride cotransport mediated disorder is a neonatal seizure, acute seizure, a chronic epilepsy, stroke, trauma, cortical malformation, CNS tumor or metabolic disorder.

4. The method of claim 3, wherein the chronic epilepsy is a chronic temporal lobe epilepsy, or chronic epilepsy related to stroke, metabolic disorder, trauma, malformation of cortical development or tumor.

5. The method of claim 1, wherein the diuretic compound is 3-(aminosulfonyl)-5-(butylamino)-4-phenoxybenzoic acid, 5-(aminosulfonyl)-4-chloro-2-[(2-furanylmethyl)-amino]benzoic acid, [2,3-dichloro-4-(2-methylene-1-oxobutyl) phenoxy]acetic acid, or a combination thereof.

6. The method of claim 5, wherein the diuretic compound is 3-(aminosulfonyl)-5-(butylamino)-4-phenoxybenzoic acid.

7. The method of claim 1, wherein said method further comprises administering a second therapeutic agent, wherein the second therapeutic agent comprises a γ-aminobutyric acid A (GABA_{A}) receptor modulator, an anticonvulsant agent, ion channel inactivator, an antidiuretic agent, or a combination thereof.

8. The method of claim 7, wherein the GABA_{A} receptor modulator comprises a GABA_{A} receptor positive allosteric modulator.

9. The method of claim 8, where in the GABA_{A} receptor positive allosteric modulator is a barbiturate or a benzodiazepine or a combination thereof.

10. The method of claim 8, wherein the GABA_{A} receptor modulator is an anticonvulsant agent.

11. The method of claim 10, wherein the anticonvulsant GABA_{A} receptor modulator is tiagabine or acetazolamide or a combination thereof.

12. The method of claim 7, wherein the antidiuretic agent is a peripherally-acting antidiuretic agent.

13. The method of claim 1, wherein the subject is human.

14. The method of claim 1, wherein the subject is a neonate.

15. A method for treating a sodium potassium chloride cotransport mediated disorder comprising administering to a subject in need of such a treatment a therapeutically effective amount of a compound capable of decreasing neuronal chloride accumulation in the subject.

16. The method of claim 15, wherein the compound comprises 3-(aminosulfonyl)-5-(butylamino)-4-phenoxybenzoic acid, 5-(aminosulfonyl)-4-chloro-2-[(2-furanylmethyl)amino] benzoic acid, [2,3-dichloro-4-(2-methylene-1-oxobutyl) phenoxy]acetic acid, or a combination thereof.

17. The method of claim 15, wherein said method further comprises administering a second therapeutic agent, wherein the second therapeutic agent comprises a γ-aminobutyric acid A (GABA_{A}) receptor modulator, an anticonvulsant agent, ion channel modulator, an antidiuretic agent, or a combination thereof.

18. The method of claim 17, wherein the GABA_{A} receptor modulator comprises a GABA_{A} receptor positive allosteric modulator.

19. A method for treating a neonatal seizure comprising administering to a subject in need of such a treatment a therapeutically effective amount of:
(i) a compound capable of decreasing neuronal chloride accumulation;
(ii) a compound capable of modulating GABA production or modulating GABA_{A} receptor activity; or
(iii) a combination thereof.

20. The method of claim 19, wherein the compound capable of decreasing neuronal chloride accumulation comprises 3-(aminosulfonyl)-5-(butylamino)-4-phenoxybenzoic acid, 5-(aminosulfonyl)-4-chloro-2-[(2-furanylmethyl)amino] benzoic acid, [2,3-dichloro-4-(2-methylene-1-oxobutyl) phenoxy]acetic acid, or a combination thereof.

21. The method of claim 19 further comprising administering a second therapeutic reagent comprising an anticonvulsant agent, an antidiuretic agent, or a combination thereof.

22. The method of claim 21, wherein the second therapeutic reagent comprises an anticonvulsant agent.

23. A method for treating a disorder mediated by excitotoxicity in the brain that is exacerbated by impaired inhibition of γ-aminobutyric acid (GABA), said method comprising administering to a subject in need of such a treatment, a therapeutically effective amount of a diuretic compound.

24. The method of claim 23, wherein the disorder comprises epilepsy, seizure, or a disease associated with a hypoxic-ischemic event.

25. The method of claim 23 further comprising administering a second therapeutic agent comprises a GABA receptor modulator, an anticonvulsant agent, an antidiuretic agent, or a combination thereof.

26. The method of claim 25, wherein the GABA receptor modulator is a GABA agonist.
